(19) United States
(12) Patent Application Publication
(10) Pub. No.: US 2020/0258598 A1
(43) Pub. Date: Aug. 13, 2020

(54) REVERSIBLY DEFORMABLE METAMATERIAL

(71) Applicant: THE ROYAL INSTITUTION FOR THE ADVANCEMENT OF LEARNING/MCGILL UNIVERSITY, Montreal (CA)

(72) Inventors: Lu LIU, Swansea, SC (US); Chuan QIAO, Montreal (CA); Haichao AN, Montreal (CA); Damiano PASINI, Montreal (CA)

(21) Appl. No.: 16/789,027

(22) Filed: Feb. 12, 2020

Related U.S. Application Data
(60) Provisional application No. 62/804,325, filed on Feb. 12, 2019.

(51) Int. Cl. G16C 20/30; G16C 60/00

(52) U.S. Cl. CPC G16C 20/30; G16C 60/00

(57) ABSTRACT

A metamaterial reversibly deformable when exposed to a temperature condition, has metaunits interconnected with one another to form a metaensemble. The metaunits include frames and cores attached to the frames, portions of the cores being free of connection with the frames. One of the frame and the core having a Young's modulus greater than that of the other and having a coefficient of thermal expansion less than that of the other. The metaensemble having a sequence code defining a target shape of the metaensemble, the sequence code including at least one geometric characteristic and at least one material characteristic of each of the frame and the core. The metamaterial with the sequence code being reversibly deformable from an initial shape to the target shape upon being exposed to the temperature condition, and back from the target shape to the initial shape upon withdrawal of the temperature condition.

### CROSS-REFERENCE

The present application claims priority on U.S. Patent Application No. 62/804,325 filed Feb. 12, 2019, the entire content of which is incorporated herein by reference.

### TECHNICAL FIELD

The present disclosure relates generally to metamaterials, and more particularly to lattice metamaterials having preprogramed thermal expansions and components made of such materials.

### BACKGROUND OF THE ART

Shape morphing exists in nature across most biological taxa. From plant tissues to bacteria, from marine animals to human tendons, natural materials feature seamlessly integrated architectures across the nano, micro and mesoscales, allowing for an impressive array of functional properties. This stands at the core of an intrinsic capacity for such natural materials to transform and adapt their morphology in response to water, light, temperature and other environmental stimuli.

In the synthetic world, on the other hand, products that can stretch and fold, pack and unpack, as well as change drastically in size, volume and/or shape are less easily achieved and represent practical challenges that our industry and society at large is called to address. Materials that can autonomously adapt their configurations to multifunction in a changing environment are desirable and represent future technology across disciplines and size scales.

The capacity of a material to shape morph in response to physical and/or chemical cues has been so far demonstrated with active materials and geometrically patterned passive solids. The former (i.e. active materials) are stimuli-responsive materials, such as shape memory hydrogels, for which responsiveness is administered by tailored chemical recipes in control of composition and arrangement of the material constituents, and dispensed through a specific fabrication process. Their success is manifest in the multitude of cue types so far used, but reversibility remains a challenge, i.e. the morphed material retains its state, and no reversal of shape is possible.

### SUMMARY

There is accordingly a need to at least partially address one or more of the above-noted challenges, by providing a passive metamaterial that may be capable of reversibly morphing in response to a non-mechanical stimulus, and in particularly in response to temperature change(s).

Here, it is demonstrated that a pair of passive solids, such as wood and silicone rubber, may be topologically arranged in a kirigami bi-material to shape-morph on target in response to a temperature stimulus. A coherent framework is introduced that may enable optimal orchestration of bi-material units that may engage temperature to collectively deploy into a geometrically rich set of periodic and aperiodic shapes that may shape match a predefined target. The results highlight reversible morphing by mechanics and geometry. This may contribute to relax the dependence of current strategies on material chemistry and fabrication.

Responsiveness to non-mechanical stimuli, such as temperature, necessitates a fine interplay between material functionalization and fabrication process, whereas geometric tessellations in unresponsive materials are confined to an applied mechanical force.

A class of passive metamaterials that react to temperature with reversible morphing is accordingly described herein.

1) Building block. A metaunit is devised to offer a geometric and deformation content much richer than all the existing ones, which can be condensed to simple bi-layer systems able mainly to bend only. The disclosed metaunit is a bi-material kirigami, which has an intrinsic versatility to break or retain symmetry on demand, thereby conferring a topological character delivering distinct floppy modes that can be tuned in magnitude and direction as desired.

2) Deformation-property profile. Routes for performance tuning and amplification in the geometry and material space are introduced and are defined by maps that unveil a direct correlation between the deformation amplitude the disclosed metaunit can offer and the geometric and material attributes of the metaunit. This strategy is the first at providing systematic means to encode morphing traits at the rank of the unit.

3) Unit aggregation. Rules for monolithic interaction between units are introduced via either the low CTE (coefficient of thermal expansion) material, or at a collection of high CTE locations. These may open the space for a rich multitude of tessellations with broad geometric diversity, periodic and aperiodic from both primitive and hybrid building blocks.

4) Genotype, phenotype and building block sequence code. These notions are first defined in the context of metamaterials to connote the string of functional information of each unit and to design collective motions that are frustration-free in both the forward and inverse problems.

5) Morphing on target. Corresponds to the ability of a metamaterial to deform in a target shape. The present framework is the first that can tailor a sequence code for frustration-free metaunits aperiodically arranged to enact morphing conformal to a freeform target.

6) Fabrication. The realization of this class of metamaterials may use a process involving cuts on a single piece of passive bi-materials. This may unleash the use of most existing technologies of fabrication, e.g. 3D printing.

The universal character of the metamaterials described herein engage two fronts: ushering a coherent framework for creating unresponsive solids to autonomously morph upon changes in environmental temperature only with no use of any external power, control and actuation; ii) unleashing the intertwined dependence of current technologies on process and chemistry, hence making fabrication compatible to almost any other techniques. Foreseeable applications are across the multidisciplinary spectrum of technology, such as shape-reconfigurable products that can be flat transported before in-situ unfolding in space and extreme climates on Earth, autonomous soft robotics, self-morphing medical devices, and conformable stretchable electronics, among several others.

Herein are presented routes to unlock reversible morphing triggered by temperature stimuli from a pair of passive solids geometrically shaped through a simple fabrication process. The disclosed platform avails theoretical, computational and experimental studies to empower the optimal orchestration of frustration-free metaunits in aperiodic metamaterials that can reversibly and autonomously morph into a geometrically rich set of complex shapes.

Here, temperature-driven morphing from a pair of passive solids, aperiodically patterned through a basic fabrication process is demonstrated. Temperature-responsive metaunits and aggregation rules that can form a variety of single-piece metaensembles, and present a coherent framework to deterministically predict and program their shape-shifting, are introduced. Soft modes of deformation individually encoded into the geometry of each metaunit are globally dispensed to generate shape morphing that can conform to a distinct number of shape targets. The present disclosure, highlighting the notion of functionality induced by the interplay between geometry and mechanics, promotes reversible shape-shifting from passive solids in aperiodic metamaterials and contributes to relaxing the dependence on the fabrication parameters and material composition.

In one aspect, there is provided a metamaterial configured to reversibly deform when exposed to a temperature condition, comprising a structure composed of a plurality of metaunits interconnected to form a metaensemble, each of the metaunits having a frame and a deformable member, extremities of the deformable member secured to the frame, the metaunits interconnected to each other to form the metaensemble, the frame having a Young's modulus greater than that of the deformable member, the deformable member having a coefficient of thermal expansion (CTE) greater than that of the frame, the metaensemble having a sequence code defined by one or more of a geometric property and a material property of the metaunits, the sequence code selected such that the metaensemble is reversibly deformable from an initial shape to a target shape upon the metaensemble exposed to the temperature condition and back from the target shape to the initial shape upon withdrawal of the temperature condition.

In another aspect, there is provided a method of producing a metaensemble including a plurality of metaunits and defining a sequence code, the metaensemble configured for reversibly deforming from an initial shape to a target shape upon exposure to a temperature condition, the method comprising: determining one or more geometric characteristics of the target shape; translating the determined geometric characteristics of the target shape into geometric characteristics of each of the plurality of metaunits forming the metaensemble; determining a change of shape of the metaensemble so that the metaensemble morphs to the target shape upon exposure to the temperature condition; determining material and complementary geometric properties of each of the metaunits based on the determined change of shape of the metaensemble; and manufacturing the metaensemble based on the determined sequence code.

In another aspect, there is provided a metaunit of a metamaterial, a number of the metaunits adapted to be interconnected together to form a metaensemble configured to reversibly deform when exposed to a temperature condition, the metaunit comprising a frame and a deformable member, extremities of the deformable member secured to the frame, the frame having a Young's modulus greater than that of the deformable member, the deformable member having a coefficient of thermal expansion greater than that of the frame.

In yet another aspect, there is provided a metamaterial configured to reversibly deform when exposed to a temperature condition, comprising a structure composed of a plurality of metaunits interconnected to form a metaensemble, the metaensemble having a sequence code defined by one or more of a geometric property and a material property of the metaunits, the sequence code selected such that the metaensemble is reversibly deformable from an initial shape to a target shape upon the metaensemble exposed to the temperature condition and back from the target shape to the initial shape upon withdrawal of the temperature condition.

In one aspect, there is provided a metamaterial configured to reversibly deform when exposed to a temperature condition, comprising a plurality of metaunits interconnected with one another to form a metaensemble, each of the metaunits having a frame and a core attached to the frame, a portion of the core free of connection with the frame to allow relative movement therebetween, one of the frame and the core having a Young's modulus greater than that of the other and having a coefficient of thermal expansion less than that of the other of the frame and the core, the metaensemble having a sequence code defining a target shape of the metaensemble, the sequence code including at least one geometric characteristic and at least one material characteristic of each of the frame and the core, the metamaterial with the sequence code being reversibly deformable from an initial shape to the target shape upon being exposed to the temperature condition and back from the target shape to the initial shape upon withdrawal of the temperature condition.

In another aspect, there is provided a method of producing a metamaterial configured to reversibly deform from an initial shape to a target shape upon exposure to a temperature condition, the metamaterial including a metaensemble formed of a plurality of metaunits each having a frame and a core attached to the frame, a portion of the core free of connection with the frame to allow relative movement therebetween, one of the frame and the core having a Young's modulus greater than that of the other and having a coefficient of thermal expansion less than that of the other of the frame and the core, the method comprising: obtaining one or more geometric characteristics of the target shape; determining a sequence code of the metaensemble such that the metamaterial deforms to the target shape upon application of the temperature condition, the sequence code including at least one geometric characteristic and at least one material characteristic of each of the metaunits of the metaensemble; and manufacturing the metamaterial based on the determined sequence code.

In yet another aspect, there is provided a metaunit for forming a metamaterial, comprising a frame and a core secured to the frame, a portion of the core free of connection with the frame to allow relative movement therebetween, one of the frame and the core having a Young's modulus greater than that of the other and having a coefficient of thermal expansion (CTE) less than that of the other of the frame and the core, the metaunit reversibly deformable from a first position to a second position upon application of a temperature condition and from the second position to the first position upon withdrawal of the temperature condition, a deformation of the metaunit upon application of the temperature condition different than that of both the frame and the core being separated from one another.

### BRIEF DESCRIPTION OF THE DRAWINGS

Reference is now made to the accompanying figures in which:

FIG. 1a is a schematic front view of a metaunit in accordance with one embodiment shown in an undeformed state;

FIG. 1b is a schematic front view of the metaunit of FIG. 1a shown in a deformed state;

FIG. 1c is a schematic front view of a metaunit in accordance with another embodiment resulting from a modification of the metaunit of FIG. 1a;

FIG. 1d is a graph illustrating a deformation-property profile of the metaunit of FIG. 1a in a material space;

FIG. 1e is a graph illustrating deformation-property profile of the metaunit of FIG. 1a in a geometry space;

FIGS. 2a to 2l are schematic front views of metaensembles created by different arrangements of the metaunits of FIGS. 1a and 1c;

FIG. 3a is a schematic front view of a metaunit in accordance with one embodiment shown in an undeformed state;

FIG. 3b is the metaunit of FIG. 3a shown in a deformed state;

FIG. 4a is a schematic front view of a metaunit in accordance with one embodiment shown in an undeformed state;

FIG. 4b is the metaunit of FIG. 4a shown in a deformed state;

FIG. 5a is a schematic front view of a metaensemble in accordance with one embodiment shown in an undeformed state, the metaensemble including a plurality of the metaunits of FIGS. 3a and 4a;

FIG. 5b is a schematic front view of the metaensemble of FIG. 5a shown in a deformed state;

FIG. 6a is a schematic view of a target domain in accordance with one embodiment;

FIG. 6b is a schematic view of the target domain of FIG. 6a superposed on an initial, off-target, phenotype;

FIG. 6c is a schematic view of a metaensemble encoded to match the target domain shown in FIG. 6a shown in an undeformed state, the metaensemble including a plurality of the metaunits of FIGS. 3a and 4a;

FIG. 6d is a schematic view of the metaensemble of FIG. 6c shown in a deformed state matching the target domain of FIG. 6a;

FIG. 7a is a schematic three-dimensional view of a metaunit in accordance with one embodiment shown in an undeformed state;

FIG. 7b is a schematic three-dimensional view of the metaunit of FIG. 7a shown in a deformed state;

FIG. 7c is a schematic three-dimensional view of a metaensemble including a plurality of the metaunits of FIG. 7a shown in an undeformed state;

FIG. 7d is a schematic three-dimensional view of the metaensemble of FIG. 7c shown in a deformed state;

FIG. 8a is a schematic three-dimensional view of a metaunit in accordance with one embodiment shown in an undeformed state;

FIG. 8b is a schematic three-dimensional view of the metaunit of FIG. 8a shown in a deformed state;

FIG. 8c is a schematic three-dimensional view of a metaensemble including a plurality of the metaunits of FIG. 8a shown in an undeformed state;

FIG. 8d is a schematic three-dimensional view of the metaensemble of FIG. 8c shown in a deformed state;

FIG. 9a is a schematic three-dimensional view of a metaunit in accordance with one embodiment shown in an undeformed state;

FIG. 9b is a schematic three-dimensional view of the metaunit of FIG. 9a shown in a deformed state;

FIG. 9c is a schematic three-dimensional view of a metaensemble including a plurality of the metaunits of FIG. 9a shown in an undeformed state;

FIG. 9d is a schematic three-dimensional view of the metaensemble of FIG. 9c shown in a deformed state;

FIG. 10a is a schematic three-dimensional view of a metaunit in accordance with one embodiment shown in an undeformed state;

FIG. 10b is a schematic three-dimensional view of the metaunit of FIG. 10a shown in a deformed state;

FIG. 11a is a schematic three-dimensional view of a metaunit in accordance with one embodiment shown in an undeformed state;

FIG. 11b is a schematic three-dimensional view of the metaunit of FIG. 11a shown in a deformed state;

FIG. 12a is a schematic three-dimensional view of a metaensemble including a plurality of the metaunits of FIG. 10a shown in a undeformed state;

FIG. 12b is a schematic three-dimensional view of the metaensemble of FIG. 12a shown in a deformed state;

FIG. 13a is a schematic three-dimensional view of a metaensemble including a plurality of the metaunits of FIG. 11a shown in a undeformed state;

FIG. 13b is a schematic three-dimensional view of the metaensemble of FIG. 13a shown in a deformed state;

FIG. 14a is a schematic three-dimensional view of a metaunit in accordance with one embodiment shown in an undeformed state;

FIG. 14b is a schematic three-dimensional view of the metaunit of FIG. 14a shown in a deformed state;

FIG. 15a is a schematic three-dimensional view of a metaunit in accordance with one embodiment shown in an undeformed state;

FIG. 15b is a schematic three-dimensional view of the metaunit of FIG. 15a shown in a deformed state;

FIG. 16a is a schematic three-dimensional view of a metaensemble including a plurality of the metaunits of FIGS. 10a and 11a shown in an undeformed state;

FIG. 16b is a schematic three-dimensional view of the metaensemble of FIG. 16a shown in a partially deformed state upon being exposed to a first temperature condition;

FIG. 16c is a schematic three-dimensional view of the metaensemble of FIG. 16a shown in a deformed state upon being exposed to a second temperature condition different than the first temperature condition;

FIGS. 17a to 17d are schematic three-dimensional views illustrating manufacturing steps of a metaensemble in accordance with one embodiment;

FIG. 18a is a schematic front view of a metaunit in accordance with another embodiment shown in an undeformed state, the high CTE material being shown with dashed lines;

FIG. 18b is the metaunit of FIG. 18a shown in a deformed state;

FIG. 19a is a schematic front view of a metaensemble including a plurality of the metaunits of FIG. 18a shown in an undeformed state, the high CTE material being shown with dashed lines;

FIG. 19b is the metaensemble of FIG. 19a shown in a deformed state;

FIG. 20 is a schematic from view of a metaensemble in accordance with another embodiment, the high CTE material being shown with dashed lines;

FIG. 21 is a schematic from view of a metaensemble in accordance with another embodiment, the high CTE material being shown with dashed lines;

FIG. 22a is a schematic three-dimensional view of a metaensemble in accordance with another embodiment shown in an undeformed state, the high CTE material being shown with dashed lines;

FIG. 22b is the metaensemble of FIG. 22a shown in a deformed state;

FIG. 23a is a schematic three-dimensional view of a metaensemble in accordance with another embodiment shown in an undeformed state, the high CTE material being shown with dashed lines;

FIG. 23b is the metaensemble of FIG. 23a shown in a deformed state;

FIG. 24a is a schematic three-dimensional view of a metaensemble in accordance with another embodiment shown in an undeformed state;

FIG. 24b is the metaensemble of FIG. 24a shown in a deformed state;

FIG. 25a is a schematic three-dimensional view of a metaensemble in accordance with another embodiment shown in an undeformed state, the high CTE material being shown with dashed lines;

FIG. 25b is the metaensemble of FIG. 25a shown in a deformed state;

FIG. 26a is a schematic three-dimensional view of a metaensemble in accordance with another embodiment shown in an undeformed state, the high CTE material being shown with dashed lines; and

FIG. 26b is the metaensemble of FIG. 26a shown in a deformed state.

### DETAILED DESCRIPTION

As noted above, shape morphing in response to an external stimulus has been pursued in synthetic analogs for a number of applications in engineering, architecture, and beyond. Existing concepts mostly engage two strategies: tailoring the composition and/or arrangement of the constituents through fabrication, and harnessing geometric patterns on flat surfaces from a single solid. The former, typical of active materials, generates mainly irreversible forms and has been demonstrated with an array of physical and chemical cues; whereas reversibility is manifest with the latter, but only in response to a mechanical input. Natural systems often exhibit an effortless propensity to shape morph in response to light, humidity and other environmental stimuli. Controlled formation of shape morphing has a number of distinct hallmarks, the most notable being spatial reconfigurability delivered post-fabrication, generation of prescribed motions, morphing induced functionalities (such as actuation, amplified extensibility, and folding), and time-dependent control of shape shifting. These along with other benefits have so far contributed to brand shape morphing as a topical theme of research with widespread promise of application across the spectrum of technology, such as autonomous robotics, smart textiles, shape-shifting metamaterials, minimally invasive devices, drug delivery, and tissue engineering.

The following definitions may apply in the present specification including claims:

Metamaterial: an artificial material with properties that do not exist in nature; these properties are due to structure and not material composition. Their name derives from the Greek word ‘meta,’ which means beyond, because these materials may have properties that extend beyond materials found naturally. A metamaterial is a material engineered to have a property that is not found in naturally occurring materials. A metamaterial may be made from assemblies of multiple elements fashioned from composite materials such as metals and plastics. The materials may be arranged in repeating patterns. Metamaterials may derive their properties not only from the properties of the base materials, but from their newly designed structures. Their precise shape, geometry, size, orientation and arrangement gives them their smart properties to achieve benefits that go beyond what is possible with conventional materials.

Metaensemble: An assembly of two or more metaunits secured to one another.

Metaunit: A building block used to create a metaensemble. The metaunit may be made using two or more different materials differing by both of their coefficient of thermal expansions (CTEs) and their Young's moduli. The metaunit may have properties when expose to a temperature change that is different that of both of the materials it includes. For instance, a thermal deflection of a metaunit may be different than that of both of the two or more materials composing the metaunit. A metaunit may be, in itself, a metamaterial since it may exhibit properties that do not exist in nature.

Active or smart material: A material able to exhibit a change in one or more properties (e.g., size, stiffness, color, etc.) in response to a stimuli (e.g., temperature variation, pressure variation, magnetic field, electric current, etc.).

Shape morphing in artificial materials has been demonstrated with a range of external stimuli and materials. Swelling, light, temperature, and other cues, are typical triggers in field-responsive solids, i.e. active materials that deform in response to an applied stimulus through physical or chemical changes occurring in their atomic or molecular structure. A material may be categorized as being “active” when it undergoes a change it its physical properties as a result of phase transformations, conformation shifts of their molecular structure and mechanochemical interactions of their constituents. Stimuli-responsive materials appear either individually, e.g. shape memory alloys, or in composite formations, e.g. hydrogel composites, with localized inclusions of material heterogeneity, gradation of particle concentrations in given directions, patterning of anisotropic materials, among others. These realizations mainly extend to materials that can be polymerized, cross-linked or formulated as customized ink of composites. For these, morphing is irreversible. In all these cases, however, morphing is strongly hardwired to the material composition and functional properties of the raw constituents, as well as their fabrication process. Passive materials that can morph in response to other than mechanical stimuli are so far inaccessible.

The present metamaterial, as will now be described below, may address at least some of these issues.

At the roots of the disclosed scheme, there are three basic notions with two reciprocal routes that may enact morphing on demand and in a reversible fashion: i) the definition of a functional metaunit, also referred to as a building block (BB), including two passive solids, capable of expressing distinct modes of deformation upon a change in temperature; ii) the assignment of a deformation-property profile to the BB, which may systematically correlate the achievable amplitude of deformation a BB can deliver to its material and geometric attributes; iii) the provision of aggregation rules to adjacent BBs, which might enable monolithic tessellations of broad geometric diversity. With these notions, access to morphing is through two ports of entry. The first promotes and predicts morphing from a predefined metamaterial architecture. The second generates a morphed state that can seamlessly match a prescribed target. More detail about the building blocks, also referred to as metaunits, about the metaensembles, which are assembly of a plurality of metaunits, and about the design of metaensembles are presented herein below.

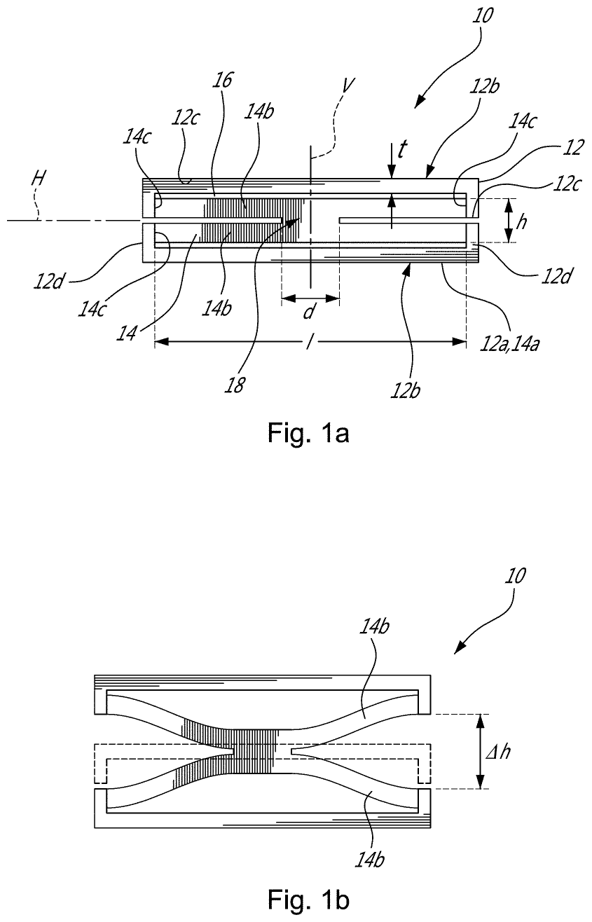

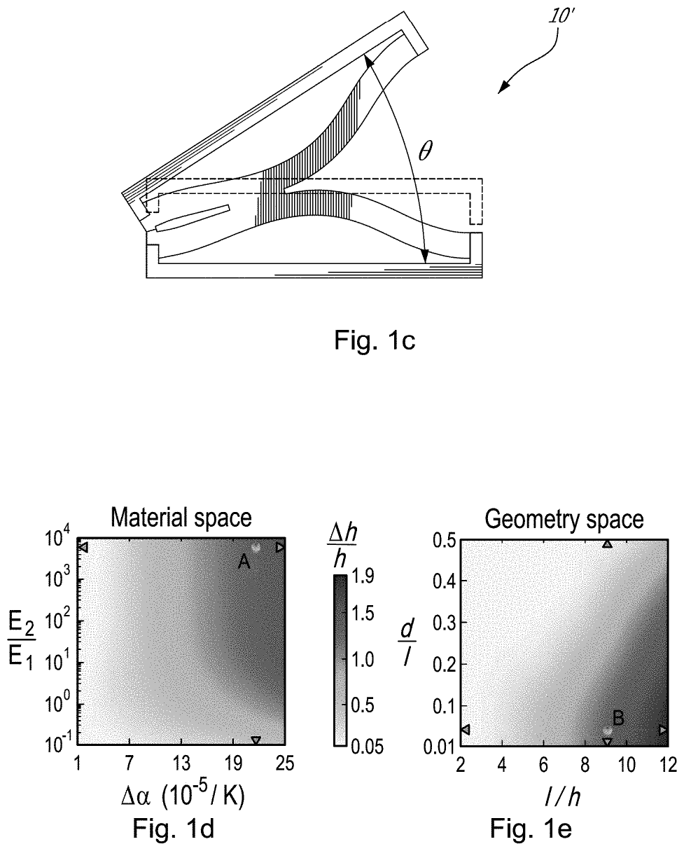

Referring now to FIG. 1a, a metaunit in accordance with one embodiment is generally shown at 10 in an undeformed state and shown in a deformed state in FIG. 1b. In the depicted embodiment, the metaunit architecture 10 includes a frame 12 with a low coefficient of thermal expansion (CTE) and a deformable member, also referred to as a core, 14 with higher CTE, each responding to temperature at a different rate. The frame 12 may be substantially rigid, at least in comparison with the deformable member 14—i.e. the frame 12 has a greater rigidity and/or stiffness and/or Young's modulus than that of the deformable member 14. As noted above, the deformable member 14 has a CTE that is greater than the CTE of the frame 12. In the embodiment shown, the deformable member 14 may be referred to as, and form at least part of, a core of the metaunit 10, as the deformable member 14 is at least partially enclosed by the frame 12. In a particular embodiment, the frame 12 substantially encloses the entirety of the deformable member 14 forming a core of the metaunit 10. The frame 12 may be capable of confining the propensity of the deformable member 14 to volumetrically expand under temperature due to their CTE mismatch. The deformable member 14 is secured to the frame 12. In the embodiment shown, vertical edges 12a, 14a of the frame 12 and of the deformable member 14 are secured to each other and may be fully bonded. However, a degree of movement is allowed between the deformable member 14 and the frame 12. Stated otherwise, at least a portion of the deformable member 14 is free of connection with the frame 12 to allow deformation of the metaunit 10.

In the embodiment shown, the frame 12 has upper and lower frame portions 12b which are identical in the embodiment shown. Each of the frame portions 12b has a central section 12c, having a thickness t, and extending along the horizontal axis H and opposite end sections 12d extending away from the central section 12c along the vertical axis V. Free ends 12e of the end sections 12d of one of the upper frame portion 12b face corresponding free ends 12e of the end sections 12d of the lower frame portion 12b. The vertical edges 12a, 14a are defined at the end sections 12d of the frame 12 to which the deformable member 14 is secured. In the embodiment shown, a slit 16 appears along an entire length of their horizontal interfaces. In other words, the central section 12c of the frame upper and lower portions 12b may be free of connection with the deformable member.

The deformable member 14 may be partially riven along its horizontal axis of symmetry H with a ligament 18 having a width d taken along the horizontal axis H. In other words, the deformable member 14 has upper and lower sections 14b secured to one another via a ligament 18. The deformable member 14 has a length l taken along the horizontal axis H. The ligament 18 connects upper and lower sections 14b of the deformable member 14 together. Each of the upper and lower sections 14b of the deformable member 14 is an elongated member extending along the horizontal axis H and having opposite ends 14c defining the vertical edges 14a, which are secured to the frame 12 as previously discussed. The deformable member 14 has a height h taken along the vertical axis V and extends between the central section 12c of the upper and lower portions 12b of the frame 12. In the embodiment shown, the height h corresponds to a distance between the two central sections 12c of the upper and lower portions 12b of the frame 12. As shown in FIG. 1b, the metaunit 10 shown is able to deform following a temperature increase and may exhibit an increase in height Δh. In the embodiment of FIG. 1a, the ligament 18 is centered relative to a center of the deformable member 14. This may yield in both the upper and lower from portions 12b to stay substantially parallel to one another when the metaunit 10 is deformed from the undeformed state of FIG. 1a to the deformed state of FIG. 1b.

Referring now to FIG. 1c, a metaunit in accordance with another embodiment shown at 10′. The metaunit 10′ differs from the metaunit 10 of FIG. 1a by having one or both of: offsetting a center of the ligament 18 from a center of the deformable member 14 and/or bonding adjacent ends 14c of the upper and lower portions 14b of the deformable member 14. In other words, the ligament 18 may be located closer to one extremity 14c of the deformable member 14 than the other. Stated differently, a center of the ligament 18 may be offset from a center of the deformable member 14.

Referring to FIGS. 1a to 1c, by harnessing the position of the deformable member ligament 18, two distinct deformation modes may be expressed with varying magnitude through temperature may be imposed to the metaunit 10. Enforced reflection symmetry with respect to a plane containing the vertical axis V imprints a unidirectional floppy mode (FIG. 1b), where the deformation of the metaunit 10 resembles an accordion that axially expands by Δh. A loss of symmetry, on the other hand, combined with end deformable member closure, may yield a metaunit 10′ having a rotational mode, where the deformation of the metaunit 10′ responds as a clothespin that can open by an angle θ. In other words, asymmetry of deformation upon exposure to a temperature condition may be imposed to the metaunit 10 (FIG. 1a) by changing a position of the ligament 18 that joins the upper and lower portions 14b of the deformable member 14.

The term “temperature condition” as used herein is understood to include, but not to be limited to, a specific temperature (e.g. a target or threshold temperature) or a change in temperature (e.g. an increase and/or a decrease). In the embodiment shown, when the metaunit 10 is not symmetrical with respect to the vertical axis V, the deformation upon the exposure to the temperature condition is also asymmetric.

Referring to FIGS. 1a-1c, the frame 12 has a frame material and the deformable member 14 has a deformable member material. The frame material has a first coefficient of thermal expansion (CTE, α) and a first Young's modulus (E) and the deformable member material has a second CTE and a second Young's modulus. In the embodiment shown, the second CTE is greater than the first CTE and the first Young's modulus is greater than the second Young's modulus. In a particular embodiment, a ratio of the Young's modulus of the first material over that of the second material is about 10. In a particular embodiment, a difference between the Young's moduli is about 90 GPa. In a particular embodiment, a difference between the CTEs is about 100 E-6/K, preferably 210 E-6/K. In a particular embodiment, whichever of the first and second materials has the highest Young's modulus has the lowest CTE and vice-versa. Other configurations and materials may be used without departing from the scope of the present disclosure. In a particular embodiment, the first and second CTEs are 10e−6/K and 110e−6/K, respectively. In a particular embodiment, the first and second Young's moduli are 110 GPa and 10 GPa, respectively. In a particular embodiment, the ratio of the Young's moduli is about 3200 and a difference between the CTEs is about 210e−6/K. Other values are contemplated.

Many factors may influence a shape of the deformed state of the metaunits 10, 10′ (FIG. 1a and FIG. 1c). These factors may include, the Young's moduli of the frame 12 and of the deformable member 14, the CTEs of the frame 12 and of the deformable member 14, a ratio of the width d of the ligament 18 to the length l of the deformable member 14; a ratio of the length l of the deformable member 14 to the height h of the deformable member 14.

There are therefore two types of factors, or parameters, influencing deformation of the metaunits 10, 10′: material parameters and geometry parameters. The Young's moduli and the CTEs are material parameters whereas the ratios of the width d to the length l and of the length l to the height h are geometric parameters. In other words, one may design a metaunit by selecting materials having given differences in their CTEs and Young's moduli and by selecting geometric parameters.

While the mode of deformation may be mainly conferred by topology (symmetrical metaunit 10 versus asymmetrical metaunit 10′), temperature, as well as materials and geometry of each metaunit 10, 10′ may govern the magnitude of the response to a temperature increase. This defines the property-deformation profile, which may be casted here in two sets. The first maps the role of materials, Δα=α2−α1 (CTE) versus E1/E2 (Young's modulus) (FIG. 1d), and the second that of geometry, d/l versus l/h (FIG. 1e), the groups of parameters that most influence BB response.

Referring now to FIG. 1d, a E2/E1 vs Δα graph illustrating the material space is shown. Each points on the E2/E1 vs Δα graph corresponds to a particular combination of Young's moduli ratio and difference in CTEs and may therefore yield a metaunit with a corresponding deformation profile.

Referring now to FIG. 1e, a d/l vs l/h graph illustrating the geometry space is shown. Similarly to the graph of FIG. 1d, each points on the d/l vs l/h graph corresponds to a particular combination of a ratio of the length d of the ligament 18 to the length l of the deformable member 14 and a ratio of the length l of the deformable member 14 and height h of said deformable member 14 and may therefore yield a metaunit with a corresponding deformation profile. To capture this dependence between topology (symmetrical vs asymmetrical metaunits), materials (Young's modulus and CTE), and geometry (d/l, h/I, etc.), one may gauge the attainable range of elastic deformation the metaunit can attain at a given temperature upon manipulation of its material and geometric attributes.

The metaunits 10, 10′ of FIGS. 1a, 1c may have difference in their CTEs of about 210×10−6/K, a ratio of their Young's moduli of about 6000; a ratio of the length l to the height h of the deformable member 14 of about 9; a ratio of the length d of the ligament 18 to the length l of the deformable member 14 of about 0.05. These parameters may correspond to points A and B on the graphs of FIG. 1d and FIG. 1e. The metaunits 10, 10′ having those properties may deform as shown in FIGS. 1b, 1c when exposed to a temperature of 120° C.

Point A on the graph of FIG. 1d correlates the amount of uniaxial deformation to a change in material properties, while point B on the graph of FIG. 1e correlates the amount of uniaxial deformation to a change in its inner architecture. While specific to this illustrative example, the property-deformation profiles may provide a systematic route to assess the deformation a BB can render at a given temperature through manipulation of its material and geometric attributes. This may be the key to predict and program morphing at the rank of the metaunit.

The terms “program”, “programmed” and “preprogrammed” as used herein in connection with the metaunits and the metamaterial formed thereby are understood to mean the selection of a specific combination of metaunits having given properties in a specific manner such that the resulting metamaterial structure, formed by the metaunits, may form a predetermined shape when one or more temperature conditions are met and is reversibly deformable between an initial shape and a predetermined target shape when exposed to a predetermined temperature condition.

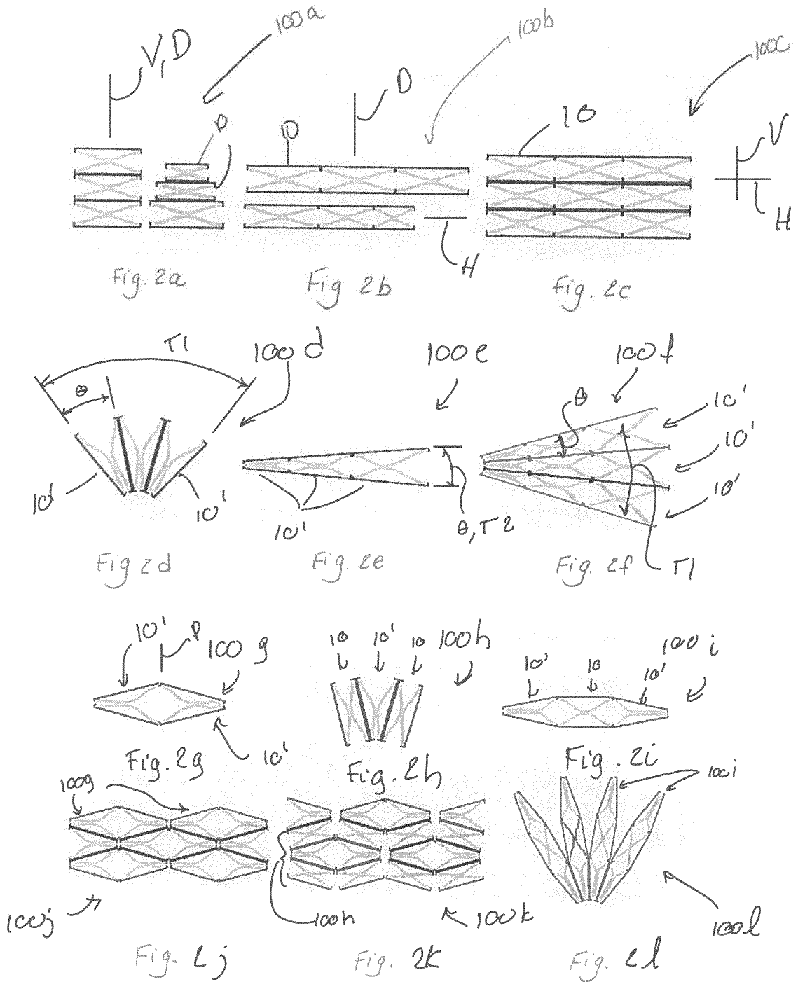

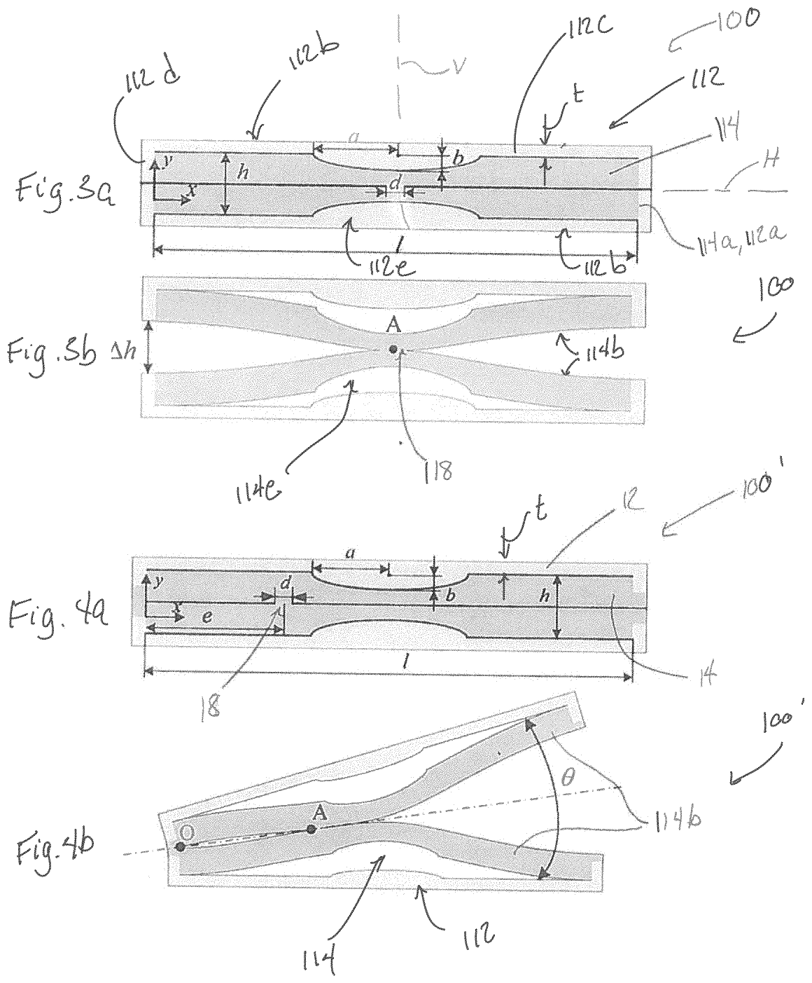

As two types of metaunits, namely the U-type metaunit 10 and the R-type metaunit 10′, have been described, reference is now made to FIGS. 2a to 2l that illustrate possible arrangement of those metaunits 10, 10′ into a plurality of metaensembles.

Referring now to FIGS. 2a to 2l, at the next level, there are metaunits aggregates which may be generated from a single piece of bi-material, a monolithic dual material panel, as opposed to an assembly of individual parts connected together. The intrinsic characteristics of metaunits are conducive to the generation of an array of metaunit aggregates with may exhibit rich geometric diversity. FIGS. 2a to 2l shows a collection of options, among others. The building blocks are shown to form spatially invariant periodic and aperiodic tessellations or metaensembles not only from primitive units, e.g. R-R or U-U, but also from hybrid cells, e.g. U-R-U, that may provide access to a diverse set of configurations. Interaction between adjacent metaunits might take place through monolithic connections that might impose the way BBs act collectively, e.g. parallel, series and combination thereof, via either the low CTE material, or at a collection of high CTE locations.

Referring more particularly to FIG. 2a, a metaensemble 100a including a plurality of metaunits 10 is shown. The metaensemble 100a is made by stacking up the metaunits 10 that expand symmetrically along their vertical axis V that is parallel to a direction of expansion D of the metaunits 10. This metaensemble 100a may be manufacture by a serial stacking of the metaunits 10 described herein above with reference to FIG. 1a. Herein, a serial stacking implies that a total elongation of the metaensemble 100a may correspond to a sum of elongations of each of the metaunits 10. In the embodiment shown, two adjacent metaunits 10 are secured to one another via the central portions 12c (FIG. 1) of their frames 12.

Referring to FIG. 2b, a metaensemble 100b including a plurality of metaunits 10 is shown. The metaensemble 100b is made by disposing the metaunits 10 along their horizontal axis H. In other words, the metaunits 10 are disposed along a direction perpendicular to their respective direction of elongation D. This configuration corresponds to a parallel stacking. Herein, a parallel stacking implies that a total elongation of the metaensemble 100b corresponds to the elongation of one of the metaunits 10. In the embodiment shown, two adjacent metaunits 10 are secured to one another via the end sections 12d of their frames 12.

Referring to FIG. 2c, a metaensemble 100c including a plurality of metaunits 10 is shown. As illustrated, the metaensemble 100c is a combination of serial and parallel stacking. A central on of the metaunits 10 may be secured to its neighbours via both of the end sections 12d and the central section 12c of their frames 12.

The metaunits 10 of the metaensemble shown in FIGS. 2a to 2c may be symmetric along two axes (vertical V and horizontal H axes). Consequently, they may retain their symmetry when expanding.

Referring now to FIGS. 2d to 2f, metaensemble may be manufacturing by combining asymmetric, or R-type, metaunits 10′ as described herein above with reference to FIG. 1c disposed in serial (FIG. 2d), in parallel (FIG. 2e), or a combination of serial and parallel (FIG. 20. Similarly to the configurations depicted above with reference to FIGS. 2a to 2c, the adjacent building blocks 10, 10′ may be secured to one another via the central section 12c, the end sections 12d, or both of the central and end sections 12c, 12d of their frames 12. The total angle of deformation T1 of the metaensemble 100d of FIG. 2d may correspond to a sum of the angle θ of deformation of each of the metaunits 10′ composing it. The total angle of deformation T2 of the metaensemble 100e may corresponds to the angle θ of deformation of one of the metaunit 10′. The metaensemble 100f corresponds to an assembly of a plurality of the metaensemble 100e described above with reference to FIG. 2e.

Referring now to FIGS. 2g to 2i, other embodiments of metaensembles are shown. The disclosed metaensemble are manufactured by the combination of symmetric 10 and asymmetric 10′ metaunits stacked up in series and in parallel. As one can imagine, a plurality of configurations are possible and are not all disclosed herein. Consequently, the scope of the present disclosure should not be limited by the disclosed examples of metaensembles.

Referring more particularly to FIG. 2g, the metaensemble 100g includes two of the metaunits 10′ described above with reference to FIG. 1c disposed symmetrically about a symmetry plane P. As shown, the deformed state of the metaensemble 100g has a diamond shape. The two metaunits 10′ may be secured to one another via the end sections 12d (FIG. 1a) of their frames 12.

Referring more particularly to FIG. 2i, the metaensemble 100h includes two U-type metaunit 10 disposed on opposite sides of an R-type metaunit 10′. The R-type metaunit 10′ is secured to its neighbouring U-type metaunits 10 via the central section 12c of their frames 12.

Referring more particularly to FIG. 2i, the metaensemble 100i includes two R-type metaunits 10′ disposed on opposite sides of a U-type metaunit 10. The U-type metaunit 10 is secured to its neighbouring R-type metaunits 10′ via the end section 12d of their frames 12. FIGS. 2j to 2k show three embodiments of metaensemble 100j, 100k, 1001 that may be obtained by assembly a plurality of the metaensemble 100g of FIG. 2g, 100h of FIG. 2h, and 100i of FIG. 2i, respectively.

It is understood that a plurality of other configurations may be obtained with any suitable combinations of U-type and R-type metaunits 10, 10′. Moreover, any of the metaensembles described above with reference to FIGS. 2a to 2l may be part of an assembly including any other of those metaensembles. In other words, a metaensemble including a combination of any of the metaensembles of FIGS. 2a to 2l may be obtained.

Referring now to FIGS. 3a and 3f, another embodiment of a U-type building block, or metaunit, is shown generally at 100. The metaunit 100 has a frame 112 and a deformable member 114 enclosed by the frame 112. The frame 112 has upper and lower sections 112a that are movable one relative to the other and secured to one another via the deformable member 114.

Each of the upper and lower sections 112b of the frame 112 has a central section 112c and opposite end sections 112d extending from opposite ends of the central section 112c toward the other of the upper and lower sections 112a.

The deformable member 114 has upper and lower sections 114b each located adjacent a respective one of the upper and lower sections 112b of the frame 112. The upper and lower sections 114b of the deformable member are secured to one another via a ligament 118. The upper and lower sections 114b of the deformable member 114 defines edges 114a at their extremities that are secured to the end sections 112d of the upper and lower sections 112b of the frame 112.

In the embodiment shown, each of the upper and lower sections 112b of the frame 112 defines a semielliptical protrusion 112e projecting toward the deformable member 114. Correspondingly, both of the upper and lower sections 114b of the deformable member 114 defines a semielliptical recess, groove, or slit, 114e configured to matingly receive a respective one of the semielliptical protrusion 112e of the frame 112. The semielliptical slit 114e may facilitate the onset of deformation.

Many parameters of the metaunit 100 may be varied. These parameters include, for instance, As length l of the deformable member 114 taken along the horizontal axis H, height h of the deformable member 114 taken along the vertical axis V, half-length a of the semielliptical protrusion/slit 112e, 114e taken along the horizontal axis H, width d of the ligament 18 taken along the horizontal axis H, and height b of the semielliptical protrusion/slit 112e, 114e, taken along the vertical axis V.

For this metaunit 100, the ligament 118 is centered. In other words, a center of the ligament 118 is coincident with the vertical axis V, which is a symmetry axis of the metaunit 100. In this case, the selection of these geometric parameters affect the expansion Δh (FIG. 3b) the metaunit 100 exhibits upon a given temperature change. It is understood that the respective Young's moduli and CTEs of both the deformable member 14 and the frame 12 may affect the expansion Δh of the metaunit 100.

Referring now to FIGS. 4a and 4b, another embodiment of a R-type building block, or metaunit, is shown generally at 100′. As shown, and as for the U-type metaunit 100 of FIG. 3a, many geometric parameters may be varied to tune the response of the metaunit 100 to a temperature variation. For the sake of conciseness, only elements of the R metaunit 100′ that differ from the U metaunit 100 of FIG. 3a are described below.

The R-type metaunit 100′ may include all of the parameters of the U-type metaunit 100 described above in reference to FIG. 3a plus a position of the ligament 118. The position of the ligament 118 may be adjusted by varying a distance e between the bonded extremities 114a of the deformable member 114 and the ligament 118 along the horizontal axis H. The distance e may extend from the bonded extremities 114a to a center of the ligament 118. In the embodiment shown, the upper and lower sections 114b of the deformable member 114 are secured to one another both via the ligament 118 and at one of their ends. Alternatively, the upper and lower sections 114b of the deformable member 114 may be secured to one another solely via the ligament 118. This may allow the metaunit 100′ to expand asymmetrically upon a temperature change. In a particular embodiment, the closer the ligament 118 is to the bonded extremities of the deformable member 114, the greater the angle θ will be exhibited by the R-type metaunit 100′ upon a temperature variation.

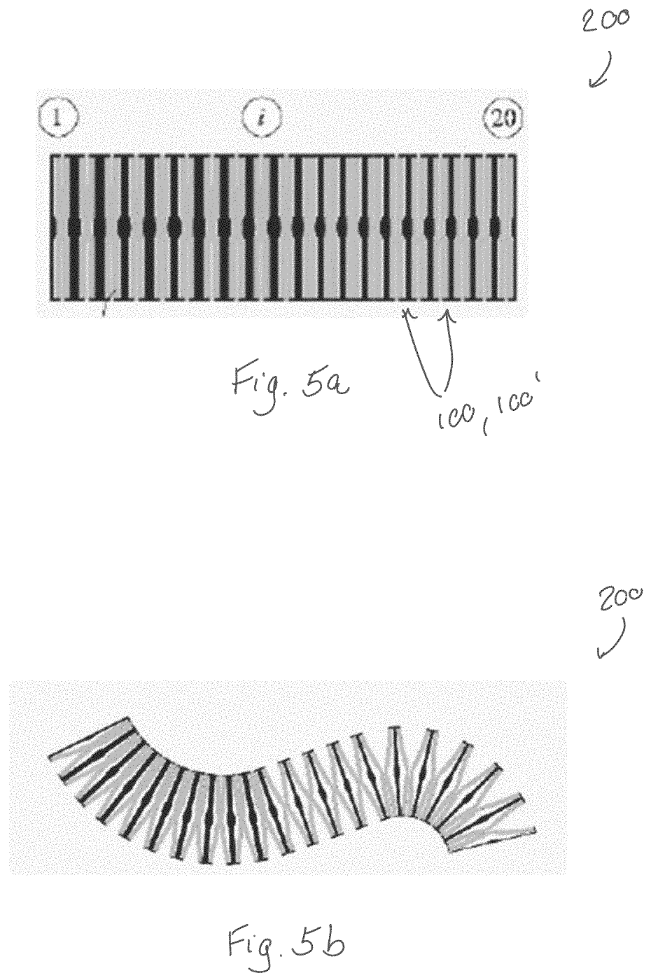

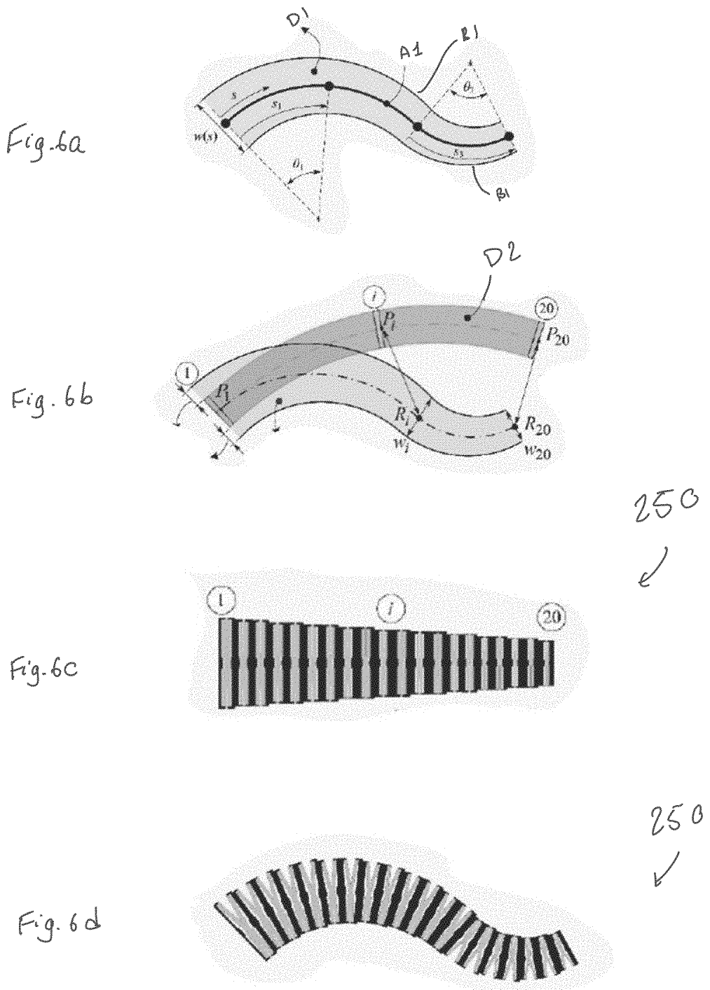

Referring now to FIGS. 5a and 5b, a metaensemble in accordance with one embodiment is shown generally at 200. The disclosed metaensemble includes a sequence of 20 metaunits 100, 100′ of a given pair of materials that may be monolithically connected in series. It is understood that more or less than 20 metaunits may be used without departing from the scope of the present disclosure.

The metaensemble 200 is shown in an undeformed state in FIG. 5a and in a deformed state in FIG. 5b. The metaensemble 200 may move from the undeformed state to the deformed state upon application of a temperature condition, such as a temperature increase or decrease, and move back from the deformed state to the undeformed state upon removal of the temperature condition, or under application of an opposed temperature condition, such as a temperature decrease of a magnitude corresponding to that of the temperature increase. The undeformed state, which may be referred to as the metamaterial genotype, may be defined by a string of information, referred to as the BB sequence code. The sequence code may be expressed as follows:

B_{t/h}^{i±(h, l/h d/l)}

Where B stands for U or R depending if the i^{th} metaunit is a U metaunit 10, 100 or a R metaunit 10′, 100′; t/h is the ratio of the thickness t of the upper and lower portions 12b, 112b of the frame 12, 112 to the height corresponding to a distance between their corresponding upper and lower portions 12c, 112c; h is the height of the deformable member 14, 114; l/h is the ratio of the length of the deformable member 14, 114 to the height h; d/l is the ratio of the width of the ligament 18, 118 to the length of the deformable member 14, 114. When “+” is used in the superscript, it implies that a direction of rotation of the R metaunit 10′, 100′ is clockwise and “−” is used when the direction of the rotation of the R metaunit 10′, 100′ is counter clockwise.

The sequence code is therefore a list of properties, both material and geometric, of each of the metaunits composing a metaensemble of a metamaterial.

It is understood that the sequence code may include more parameters, these parameters may include, for instance, dimensions of the semielliptical slit 114e, the position e of the ligament 18, 118, ratio of the half-length a of the semi-elliptical slit 114e to the height b of said slit 114e, ratio of the position e of the ligament to the width d of the ligament, ratio of the width d of the ligament to the half-length a of the semi-elliptical slit 114e, and so on.

With the notions discussed above, the morphing problem of a single piece ensemble of metaunits 10, 10′, 100, 100′ along to two pathways addressing the questions: how to predict, and how to program global transformations, is tackled. The goal may be to predict the morphed shape of a metaensemble upon a cycle change of temperature (e.g., application of a temperature condition).

The sequence code discussed above may carry the order and functional instructions that may enable cooperative, frustration-free, shape changes with closely matched deformation at the BB interfaces; it may fully connote the collective deformed state of the metamaterial, physically expressed by the phenotype. In other words, the phenotype may correspond to the shape of the metaensemble after deformation induced by the application of, for instance, a temperature condition.

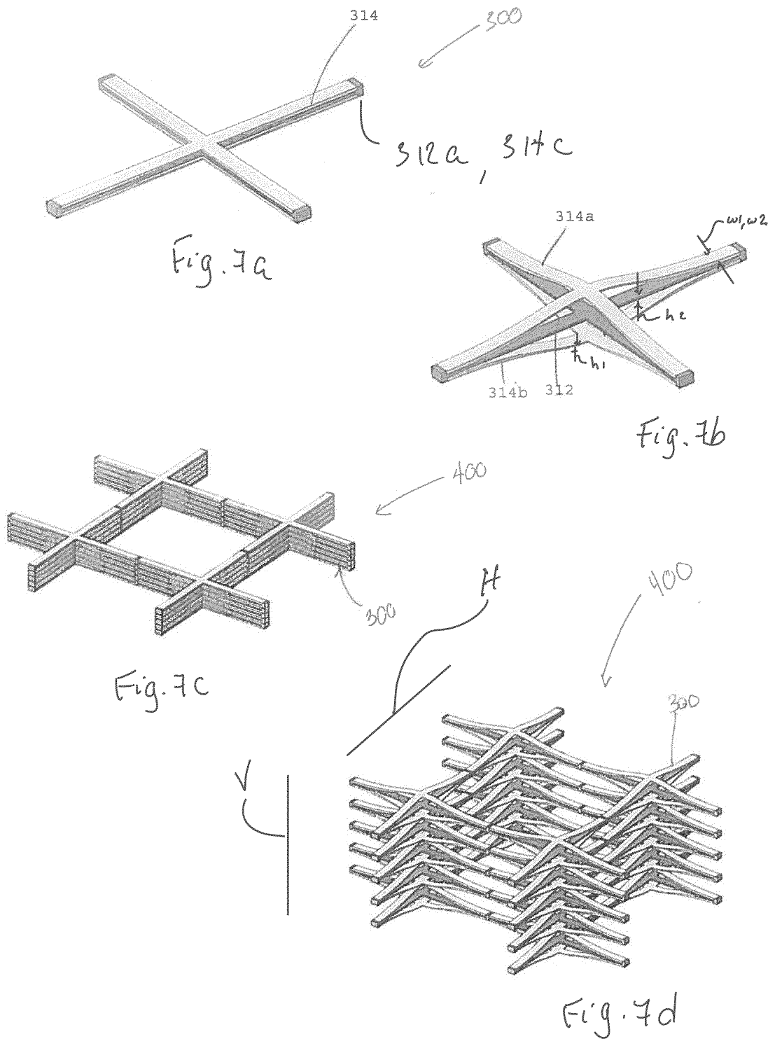

Referring now to FIGS. 6a to 6d, the complimentary route is depicted with another illustrative example in which the goal may be to program the genotype with a BB sequence code that elicits shape-shifting into a phenotype matching a given target. The target shape is shown in FIG. 6a. In the embodiment shown, two main steps are involved: extraction and translation. The extraction step may involve using the shape descriptors of the target domain D1, described here with a central axis A1 and two symmetric boundaries B1 of varying width w(s); the width w(s) being a distance between the two boundaries B1. The translation step may use the target descriptors obtained from the target domain D1 to decode a tailored BB sequence for a phenotype that may conform to the target.

To do so, the morphed configuration of an off-target phenotype D2 is used. The off-target phenotype D2 may be assigned with an arbitrary sequence of BBs, conformal to the target domain; this may be done by minimizing the gaps between their central axes and their unmatched widths w(s). The result may be a tailored BB sequence code that may enact morphing on target upon heating and directs a reversal upon cooling.

In a particular embodiment, a sequence code may be obtained from a desired phenotype or deformed shape. From the desired shape, an initial sequence listing is obtained and the different parameters of the sequence code described above may be iteratively changed until a genotype sequence code is obtained and that a metaensemble 250 manufactured using this sequence code, upon application of a temperature condition, may deform to a deformed shape (FIG. 6d) matching the target domain and revert back to its initial, undeformed shape (FIG. 6c), upon withdrawal of the temperature condition.

Stated differently, a metaensemble may include a plurality of metaunits interconnected to one another. They may be connected by their frames or by their deformable members. Each of the metaunits may have their respective geometric and material properties (the sequence code), such that the metaensemble is deformable from an initial shape (also referred as the genotype) to a target, or deformed, shape (also referred to as the phenotype) upon the metaensemble exposed to the temperature condition. The metaensemble may deform back from the target shape to the initial shape when the temperature condition is withdrawn. The sequence code is determined such that the resultant metaensemble is deformable to match the target shape when exposed to the temperature condition.

The response to temperature of the disclosed morphable materials may be programmed such that adjacent units may act collectively to reconfigure into a desired form. Here, the target to match is a domain (FIG. 6a) with a central axis, an arc spline consisting of G1 continuous arcs and straight segments, and two boundaries that are symmetric and continuous with varying width. The target may be matched by first enforcing equality constraints to guarantee frustration-free motions between adjacent units and inequality constraints that restrict BB deformation within feasible ranges. These conditions may be framed into a constrained optimization problem that may mathematically restructure the string of information contained in the BB sequence code of an un-programmed (off-target) phenotype, which may be far from the target because it is randomly assigned with an arbitrary sequence of BBs. Because the central axis and boundaries of the off-target phenotype are incompatible with those of the target domain, the sum of the squares of the distance between their central axes and the mismatched widths of their boundaries is minimized.

The frame 12, 112 may be made of hardwood (e.g., black walnut panel, Midwest Products Co., USA) and the deformable member 114 may be made of an elastomer (R-2374A silicon rubber compound, Silpak Inc., USA). It is understood that the metaunits may be made of other materials than those recited above and may be bigger/smaller than the dimension recited above without departing from the scope of the present disclosure.

In a particular embodiment, the disclosed framework may deterministically predict and precisely impart morphing into a single-piece metamaterial upon a change in the surrounding temperature. The match of the morphed phenotype to a target domain might be accurately controlled in space through the tailored decoding of the BB sequence of its genotype. The constitutive solids may be passive, yet their topological arrangement into the planar metaunit might form functional aperiodic aggregates that might yield giant shape-shifting of broad geometric diversity.

Overall, the disclosed framework may avail a fine interplay between geometry and mechanics of dual material metaunits to enact shape morphing in their monolithic ensemble. It may predict local and global morphing, as well as generate aperiodic architectures that can transform into predefined planar and spatial targets. Reversibility through temperature may be one of its assets, followed by the passive nature of the solids, which may cut the need for external power, control, and actuation. Other pairs of passive solids including metals might be used, as long as they offer a sizable distinction in CTE. Purposely implemented with simple yet efficient means of fabrication, the disclosed platform may be well-suited to other fabrication technologies, e.g. multi-material 3D printing, offers routes for upscaling and downscaling as dictated by the application, and can be extended to account for three-dimensional units. Overall, the present disclosure may expand and complement the capabilities of existing approaches and technologies; shape-shifting is a functionality that appeal to multiple sectors across disciplines, especially where folding, packaging, and conformational changes are paramount requirements to meet, such as self-reconfigurable medical devices and drug delivery systems, autonomous soft robotics, reversible self-deployment and in-situ folding in extreme climates on Earth and in space, and conformable stretchable electronics.

Producing a metamaterial configured to reversibly deform from an initial shape to a target shape upon exposure to a temperature condition may include: obtaining one or more geometric characteristics of the target shape; determining a sequence code of the metaensemble such that the metamaterial deforms to the target shape upon application of the temperature condition, the sequence code including at least one geometric characteristic and at least one material characteristic of each of the metaunits of the metaensemble; and manufacturing the metamaterial based on the determined sequence code.

In the embodiment shown, determining the sequence code includes: a) selecting first values of the sequence code; b) obtaining a model of the metamaterial based on the first values of the sequence code; c) simulating a deformation of the model of the metamaterial upon exposure to the temperature condition; d) determining second values of the sequence code in function of a difference between the simulated deformation of the model of the metamaterial and the target shape; and e) repeating steps b) to d) until the simulated deformation of the model matches the target shape.

Determining the sequence code may include determining Young's moduli, CTEs, and dimensions of each of the frames and the cores of each of the metaunits. Obtaining one or more geometric characteristics of the target shape includes modeling the target shape as a target domain with a central axis with upper and lower boundaries.

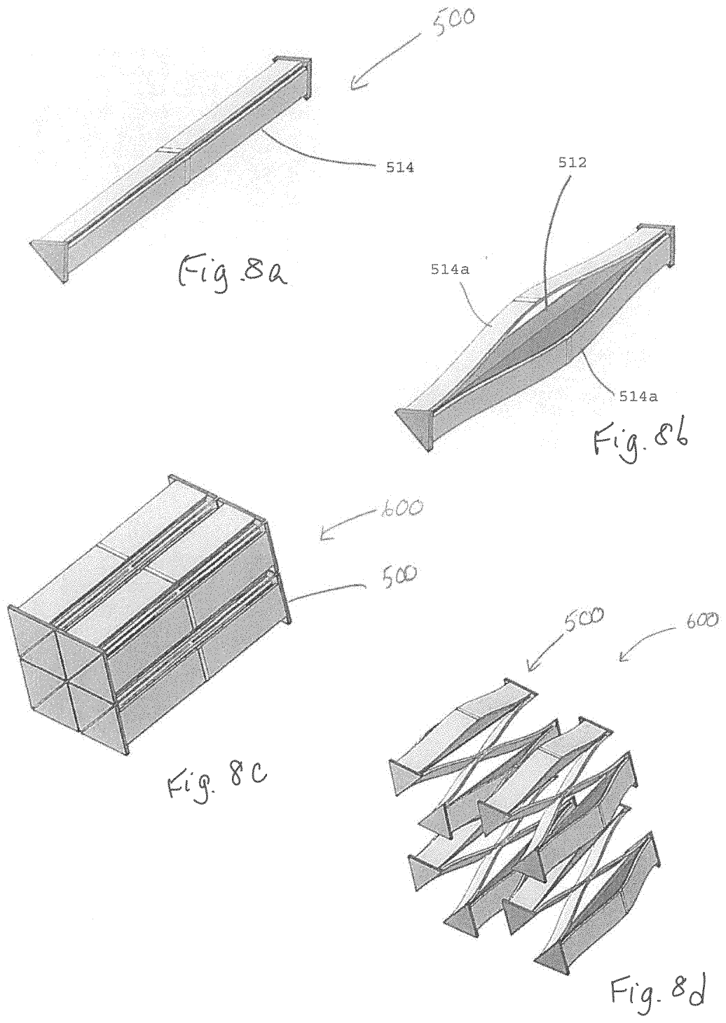

Other embodiments of metaunits are described herein above with reference to FIGS. 7a to. The metaunits may be assembled in any suitable way. Any combination of the metaunits disclosed herein may be used to create a metaensemble.

Referring to FIGS. 7a and 7b, a metaunit in accordance with an embodiment is shown at 300 in an undeformed state (FIG. 7a) and in a deformed state (FIG. 7b). The metaunit 300 includes a frame 312 and a deformable member 314 at least partially enclosing the frame 312. Herein, enclosed implies that the deformable member 314 has at least two portions 314a, 314b and the frame 312 is located between the at least two portions 314a, 314b of the deformable member 314.

In the embodiment shown, the frame 312 and the deformable member 314 are both X-shaped. Extremities 312a of the frame 312 are secured to extremities 314c of the deformable member 314. In the embodiment shown, the deformable member 314 is free of connection to the frame 312 but for its extremities 314c.

The frame 312 of the present metaunit 300 is made of a material having a CTE lower than that of the deformable member 314 and a higher Young's modulus than that of the deformable member 314. Upon exposure to a temperature increase, upper and lower frame sections 314a, 314b extend away from each other at locations where they are not connected to the frame 312.

Each of the deformable member 314 and the frame 312 may have its respective thickness h1, h2 and width w1, w2, which may be equal or different and which may be tailored as described above in a given sequence code.

Referring to FIGS. 7c and 7d, a metaensemble 400 is shown in an undeformed state (FIG. 7c) and in a deformed state (FIG. 7d). The metaensemble 400 includes a plurality of metaunits 300 as described herein above with reference to FIGS. 7a, 7b. The metaensemble 400 is made by stacking up the metaunits 300 both in serial along a vertical axis V and in parallel along a horizontal axis H. The metaunits 300 are connected to each other via their deformable member 314. Junction points between the metaunits 300 may be offset from a center of the X-shaped deformable member 314 so that the metaensemble 400 may deform asymmetrically upon an increase in temperature. Two units 300 disposed in series may be secured to one another via their deformable member whereas two units 300 disposed in parallel may be secured to one another via their frame.

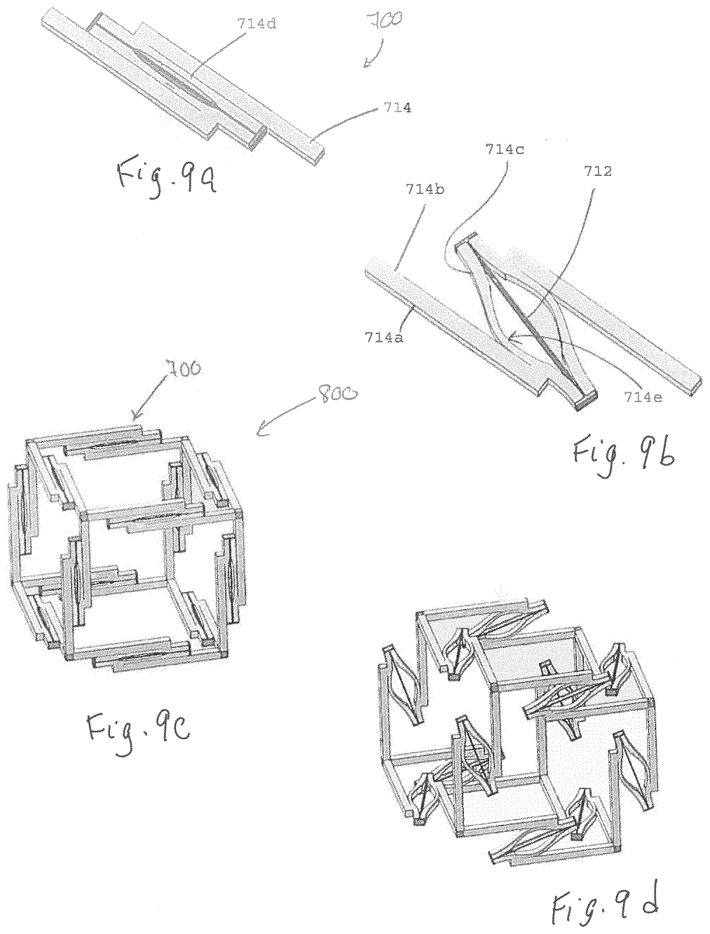

Referring to FIGS. 8a and 8b, another embodiment of a metaunit is shown at 500 in an undeformed state (FIG. 8a) and in a deformed state (FIG. 8b). The metaunit 500 includes a frame 512 enclosed by a deformable member 514. The frame 512 is a triangular prism and the deformable member 514 has three deformable member portions 514a connected to the frame 512 at their respective extremities; each of the deformable member portions 514a facing a rectangular face of the frame 512. In the embodiment shown, the frame 512 is made of a material having a Young's modulus greater than that of the deformable member 514 and having a CTE less than that of the deformable member 514. The frame and deformable member may be secured to one another at their respective extremities.

Different parameters such as the width and thickness of the frame and of the deformable member may be parameters used in a sequence code as described herein above.

Referring to FIGS. 8c and 8d, a metaensemble 600 is shown in an undeformed state (FIG. 8c) and in a deformed state (FIG. 8d). The metaensemble 600 includes a plurality of metaunits 500 as described herein above with reference to FIGS. 5a and 5b. The metaunits 500 are connected to each other via their deformable member 514. Junction points between the deformable members 514 of the metaunits 500 may be offset from a center of the frame 512 so that the metaensemble 600 may deform asymmetrically when exposed to a temperature increase. A position of the junction points may be a parameter encoded in the sequence code.

Referring now to FIGS. 9a and 9b, another embodiment of a metaunit is shown at 700 in an undeformed state (FIG. 9a) and in a deformed state (FIG. 7b). The metaunit 700 includes a frame 712 enclosed by a deformable member 714. The frame 712 may be an elongated strip and the deformable member 714 has two deformable member portions 714a connected to the frame 712 at its extremities; each of the deformable member portions 714a facing a face of the frame 712. In the embodiment shown, the frame 712 is made of a material having a Young's modulus greater than that of the deformable member 714 and having a CTE less than that of the deformable member 714.

In the embodiment shown, each of the deformable member portions 714a has a first section 714b and a second section 714c secured to the first section 714b. The frame 712 is secured to extremities of the second sections 714c of the deformable member portions 714a. In a particular embodiment, the first and second sections 714b, 714c are defined by cutting a slit 714d in the material of the deformable member 714. Upon deformation following an increase in temperature, the first sections 714b of the two deformable member portions 714a remain parallel to each other. In the embodiment shown, the second sections 714c of the deformable member portions 714a have a sections 714e having a thickness less than a remainder of the second sections 714c. The thinning sections 714e are centered on the second sections 714c. It might be possible to change a location of the thinning sections 714e and/or to change a location of a junction between the first and second sections 714b, 714c so that the first sections 714b of the two deformable member portions 714a become non-parallel upon deformation of the metaunit 700.

Referring now to FIGS. 9c and 9d, a metaensemble 800 is shown in an undeformed state (FIG. 8a) and in a deformed state (FIG. 8b). The metaensemble 800 includes a plurality of metaunits 700 as described herein above with reference to FIGS. 9a and 9b. The metaunits 700 are connected to each other via their deformable members 714, more specifically by extremities of their respective first sections 712b of their deformable member portions 714a. Different parameters such as the width and thickness of the frame and of the deformable member may be parameters used in a sequence code as described herein above.

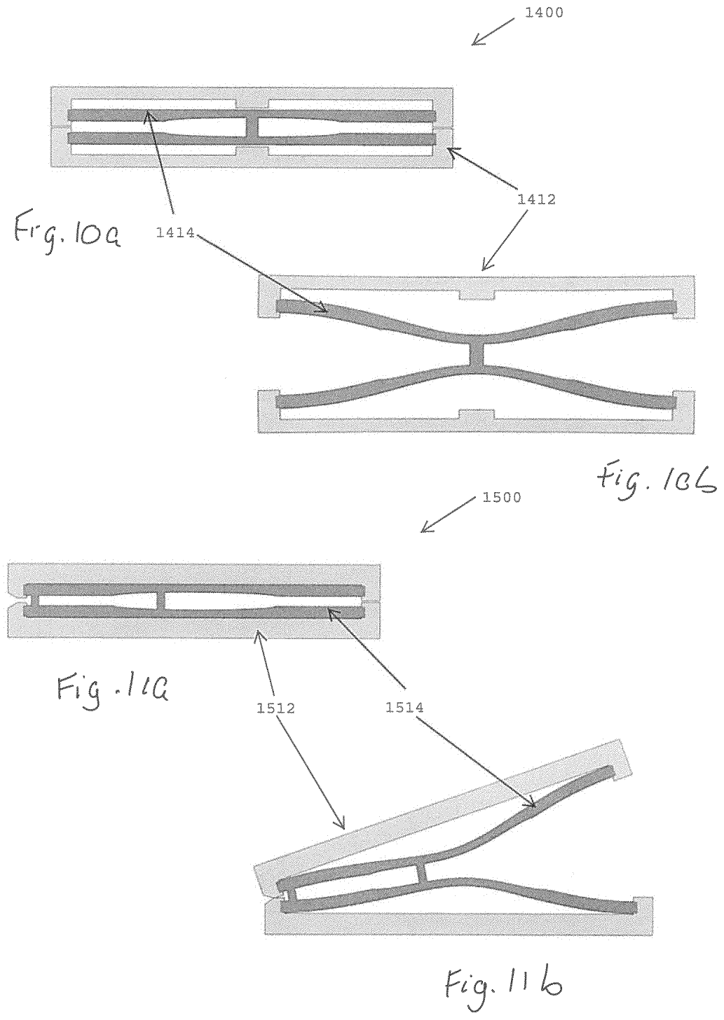

Referring now to FIGS. 10a and 10b, another embodiment of a metaunit is shown at 1400 and includes a frame 1412 and a deformable member 1414 enclosed by the frame 1412. The metaunit 1400 is similar to the metaunit 10 described herein above with reference to FIG. 1a. However, the metaunit 1400 is a snap through unit. The snap through unit 1400 is able to display an abrupt deformation at a transition temperature.

In other words, the metaunit may have a tailored geometry such that it can elicit thermal snap-through. This means that the structure may morph smoothly until it reaches a given (“programmed” or predetermined) temperature, at which it may jump to another state abruptly. This functionality can transfer to the metamaterial having a plurality of meta units.

Referring now to FIGS. 11a and 11b, another embodiment of a metaunit is shown at 1500 and includes a frame 1512 and a deformable member 1514 enclosed by the frame 1512. The metaunit 1500 is similar to the metaunit 10′ described herein above with reference to FIG. 1a. However, the metaunit 1500 is a snap through unit. The snap through unit 1500 is able to display an abrupt deformation at a transition temperature. In the embodiment shown, the snap through unit 1500 deforms asymmetrically and creates an angle between two members of the frame 1512.

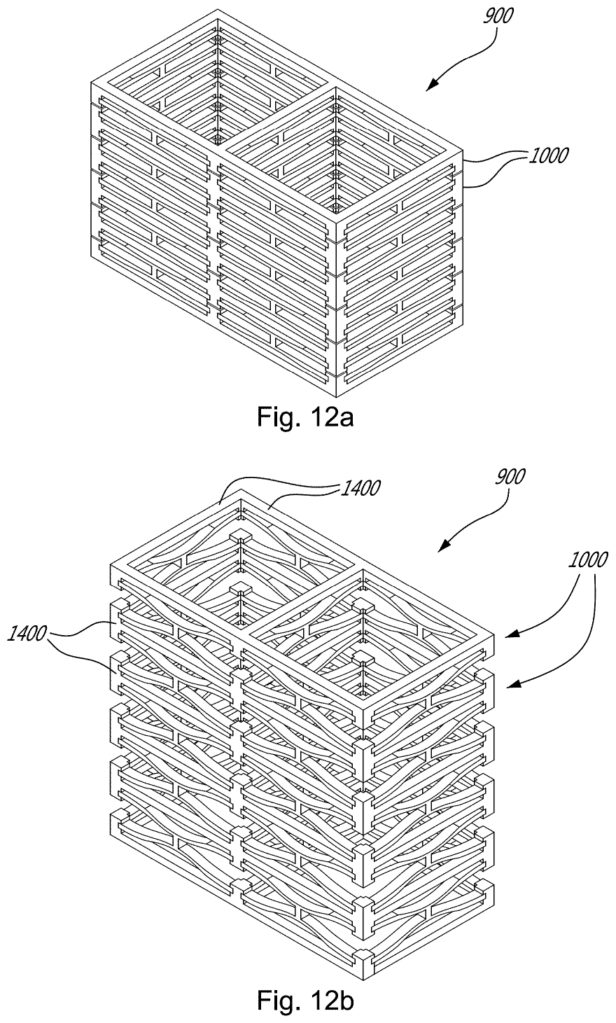

Referring now to FIGS. 12a and 12b, another embodiment of a metaensemble is shown at 900 in an undeformed state (FIG. 12a) and in a deformed state (FIG. 12b). The metaensemble 900 is created by assembly a plurality of metaunits 1000, each of which being created by an assembly of four of the metaunits 1400 described herein above with reference to FIG. 10a. More specifically, each of the metaunits 1000 includes four of the metaunits 1400 described with reference to FIG. 10a connected by their frames at their respective extremities. As shown, the metaensemble 900 includes the metaunits 1000 disposed both in serial and in parallel. Other configurations are contemplated.

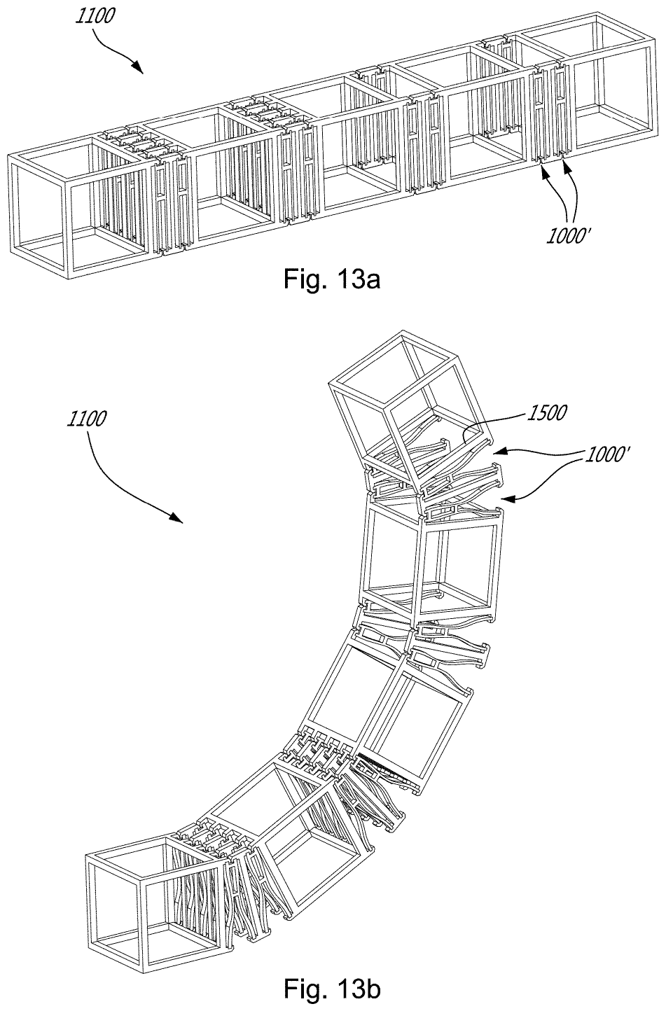

Referring now to FIGS. 13a and 13b, another embodiment of a metaensemble is shown at 1100 in an undeformed state (FIG. 13a) and in a deformed state (FIG. 13b). The metaensemble 1100 is created by assembling a plurality of metaunits 1000′ each of which being created by an assembly of four of the metaunits 1500 described herein above with reference to FIG. 11a. More specifically, each of the metaunits 1000′ includes four of the metaunits 1500 described with reference to FIG. 11a connected by their frames at their respective extremities. As shown, the metaensemble 1100 includes the metaunits 1000′ disposed in serial. Other configurations are contemplated.

Referring now to FIGS. 14a and 14b, another embodiment of a metaunit is shown at 1200. The metaunit 1200 is similar to the metaunit 300 described above with reference to FIG. 7a, but is asymmetric. The metaunit 1200 includes a frame 1212 and a deformable member 1214. In the embodiment shown, the deformable member 1214 includes two deformable member portions 1214a disposed on opposite sides of the frame 1212. In the embodiment shown, the frame 1212 and the deformable member 1214 are both X-shaped. Extremities of the frame 1212 are secured to extremities of the deformable member 1214. In the embodiment shown, the deformable member 1214 is free of connection to the frame 1212 but for its extremities.

As illustrated, each of the frame 1212 and of the deformable member 1214 includes two elements that are interconnected between their extremities and a connection point P. In the embodiment shown, the connection point P is distanced from a center of the two elements. The metaunit 1200 is able to be connected to adjacent metaunits at a junction point J that is aligned with the connection point P so that deformation upon a temperature variation creates an angle between two adjacent metaunits 1200. In other words, the asymmetry in the central node of the “X” will generate rotation on a plate put on top.

Referring to FIGS. 15a and 15b, another embodiment of a metaunit is shown at 1300. The metaunit 1300 is similar to the metaunit 300 described above with reference to FIG. 7a, but may deform asymmetrically upon a temperature variation. The metaunit 1300 includes a frame 1312 and a deformable member 1314. In the embodiment shown, the deformable member 1314 includes two deformable member portions 1314a disposed on opposite sides of the frame 1312. In the embodiment shown, the frame 1312 and the deformable member 1314 are both X-shaped. Extremities of the frame 1312 are secured to extremities of the deformable member 1314. In the embodiment shown, the deformable member 1314 is free of connection to the frame 1312 but for its extremities.

As illustrated, each of the frame 1312 and of the deformable member 1314 includes two elements that are interconnected between their extremities at a connection point P′ that is located at a center of the two elements. In the embodiment shown, each of the deformable member portions 1314a includes a junction point J′ configured to be secured to a deformable member portion of an adjacent metaunit. The junction points J′ are offset from the center of the two elements such that deformation upon a temperature variation creates an angle between two adjacent metaunits 1300. In other words, the “X” is symmetric but the edge to which a plate can be attached is offset. Then the plate would rotate.

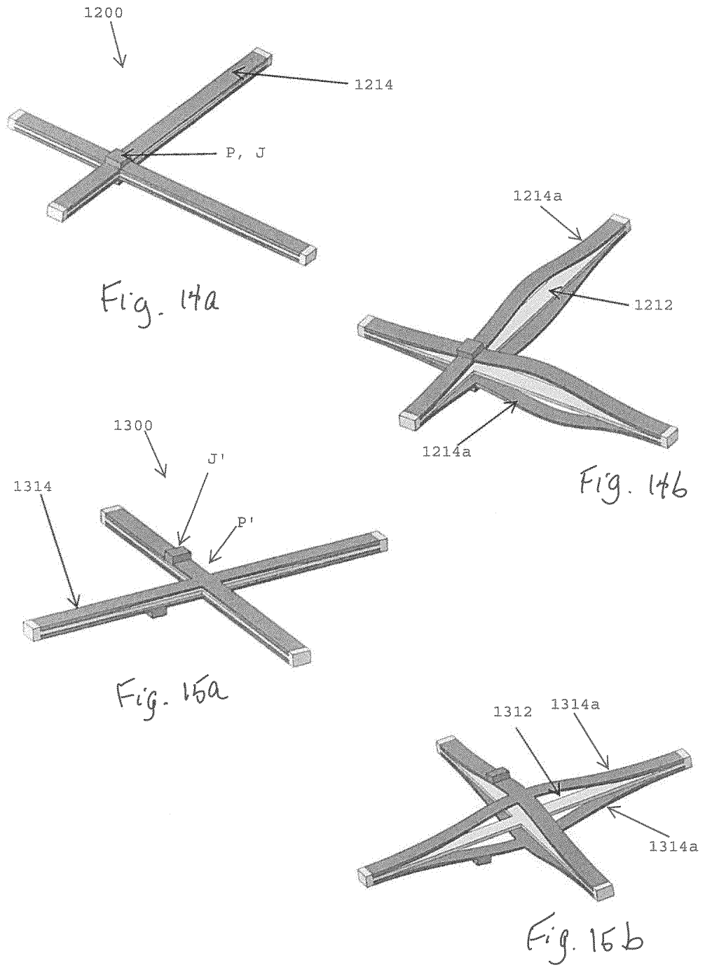

Referring now to FIGS. 16a to 16c, another embodiment of a metaensemble is shown at 1600. The metaensemble 1600 includes a plurality of metaunits 1400 and 1500 described above in reference to FIGS. 10a and 11a.

The metaensemble 1600 displays a multistate morphing caused by some units that will snap-through at a first temperature (FIG. 16b) and yield the configuration of FIG. 16c at a second temperature greater than the first temperature. In other words, the metaensemble 1600 may have a plurality of configurations dependent of the temperature it is subjected to.

Multistage or multistep morphing might be programmed via snap-through metaunits as described above and located in given position of the metamaterial. In a particular embodiment, the metamaterial might have multiple configurations in which it can work.

It is understood that other configurations of metaunits are contemplated without departing form the scope of the present disclosure. A metaensemble may include a plurality of any of the metaunits described herein above. Geometric (e.g., thickness, length, width, height, etc.) as well as material characteristics (e.g., Young's modulus and CTE) may be selected for each of the metaunits of the metaensemble to allow the metaensemble to deform in a target shape upon application of a temperature condition and to revert to its initial shape upon removal of the temperature condition.

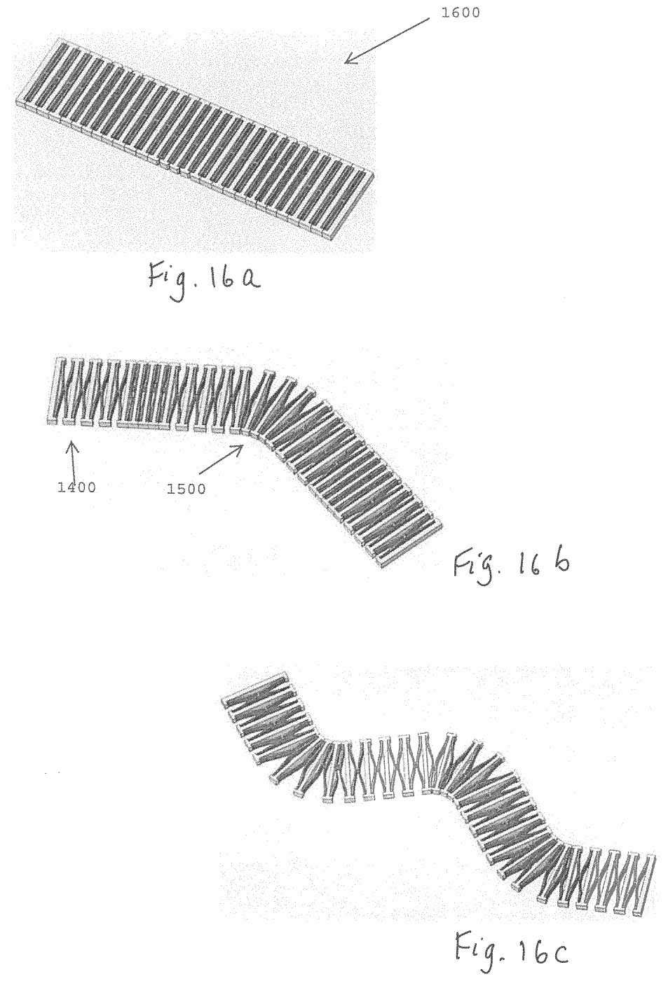

Referring to FIGS. 17a to 17d, a method of manufacturing a metaensemble in accordance with a possible embodiment is described. The fabrication process might release the dependence of metamaterial functionality from manufacturing technology and material chemistry. FIGS. 17a to 17d show the steps describing the realization of an illustrative sample comprising 3 by 5 metaunits 100 (FIG. 3a), which, in the embodiment shown, are made of a silicone elastomer (R-2374A silicone rubber compound, Silpak Inc., USA) and hardwood (Black walnut panel, Midwest Products Co., USA), the former representing the high CTE material and the latter the low CTE material. A periodic array of 15 voids aggregated in a hybrid arrangement (3 columns of units in parallel, each with 5 units connected in series), may be laser cut (CM 1290 laser cutter, SignCut Inc., CA) from a ⅛-inch-thick hardwood panel to create a void-patterned mould subsequently bonded (Instant Adhesive CA4, 3M Inc., USA) onto a ⅛-inch-thick acrylic substrate (McMaster-Carr, USA). Each void may be shaped to host the characteristic geometry of the unit deformable member featuring a semielliptical groove on both its upper and lower edges. The silicone elastomer in liquid form may be mixed with a platinum-based catalyst to create a cross-linking reaction and then injected to entirely fill the voids of the wooden array. The curing process may be performed at room temperature for about 24 hours and may turn the silicone elastomer of the building block (BB) deformable member 114 (FIG. 3a) from a liquid into solid. During the process, the silicone elastomer may bond to the wooden frame. This may offer the adequate strength for the formation of a monolithic bi-material panel. A laser cutter may perforate a set of slits into the bi-material panel, a step that may precedes the sample detachment from the substrate.

Stated differently, a meta ensemble may be manufactured by removing matter from a substrate of a first material; filling cavities created by the removal of the matter with a second material different than the first material; by separating the first and second materials at certain locations; and by creating slits in the second material. The steps illustrated in FIGS. 17a to 17d may be applied to manufacture the metaunits of FIG. 3a. The substrate of the first material may define the frame 112 and the second material may define the deformable member 114. The first and second materials are separated from one another but for at their extremities 112a, 114a. And, the ligament 118 is created by cutting slits into the deformable member 114. The slits are also defined in the frame 112 to create the upper and lower frame sections 112b to allow expansion/contraction of the metaunits.

While this metaensemble may become periodically porous with thermal response governed by a single unit, the disclosed fabrication process may enable the straightforward production of aperiodic kirigami bi-materials with global morphing controlled by the collective response of all the units.

It is understood that other materials and other manufacturing processes are contemplated without departing from the scope of the present disclosure. For instance, the metaunits described herein may be manufactured by 3D printing or any other suitable process.

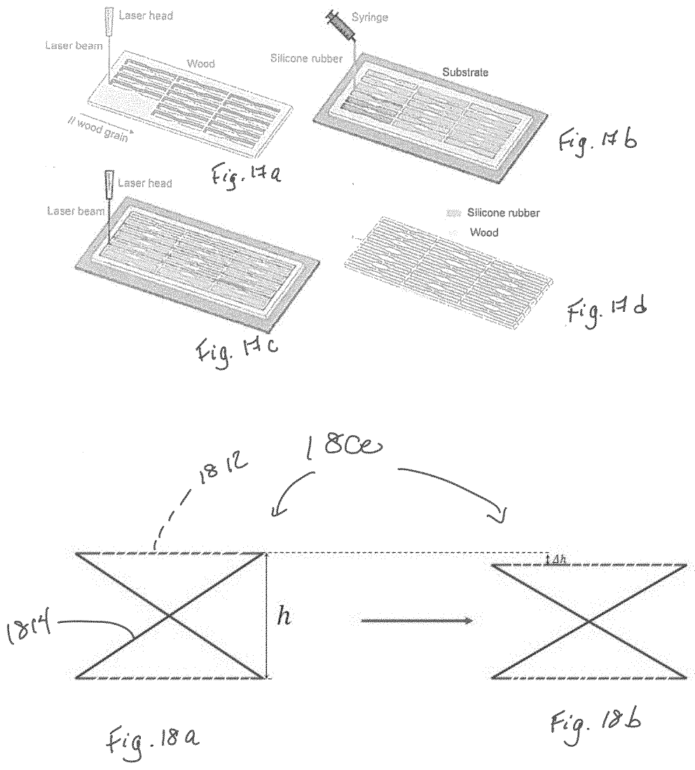

For FIGS. 18a to 26b, the material having the greater CTE is shown in dashed lines.

Referring now to FIGS. 18a and 18b, a metaunit in accordance with another embodiment is shown at 1800. In the embodiment shown, the metaunit 1800 exhibit a decrease in height Δh upon an increase of the temperature. In the metaunit 1800, the material having a high CTE is shown in dashed lines whereas the material having a low CTE is shown in solid lines. The high CTE may be 210×10e−6/K and the low CTE may be 10×10e−6/K. Other values are contemplated. The metaunit 1800 may have a frame 1812 made of a material having a CTE greater than that of a material of the core 1814.

Such a metaunit 1800 may be used in biomedical applications. For instance, this concept may be used as a contractible bandage that from a low temperature (e.g. 0 degree) could be placed on a wound at body temperature. As a result the bandage may shrink. This may reduce bandage porosity and may exert contracting forces that may enable wound closure. This may help a healing process.

Referring now to FIGS. 19a and 19b, a metaensemble including a plurality of the metaunits 1800 described above with reference to FIG. 18a is shown generally at 1900. As shown, the metaunits 1800 are assembled both in series about a vertical axis V and in parallel about a horizontal axis H. The metaunits 1800 are secured to one another via their frames 1812. FIG. 19b shows that, upon an increase in temperature, the metaensemble 1900 exhibit a contraction and decreases in its height.

Referring now to FIG. 20 a metaensemble in accordance with another embodiment is shown generally at 2000. The metaensemble 2000 may be a fractal-type metaensemble in that hierarchical arrangements of metaunits at multiple hierarchical order are possible. This may allow an amplification of the deformation. In the embodiment shown, the frame 2012 of one metaunit 2010 of the metaensemble 2000 includes itself metaensemble 2020 including a plurality of metaunits 2022. Depending of the hierarchical level of the metaensemble, the frame or deformable member of the metaunit 2022 of the metaensemble 2020 may be itself composed of an assembly of metaunits, and so on.

Referring now to FIG. 21, another embodiment of a metaensemble is shown at 2100. As shown, each of the deformable member 2114 of the metaunits may be itself composed of a metaensemble. This kind of hierarchical arrangements of units may be possible for deformation amplification.

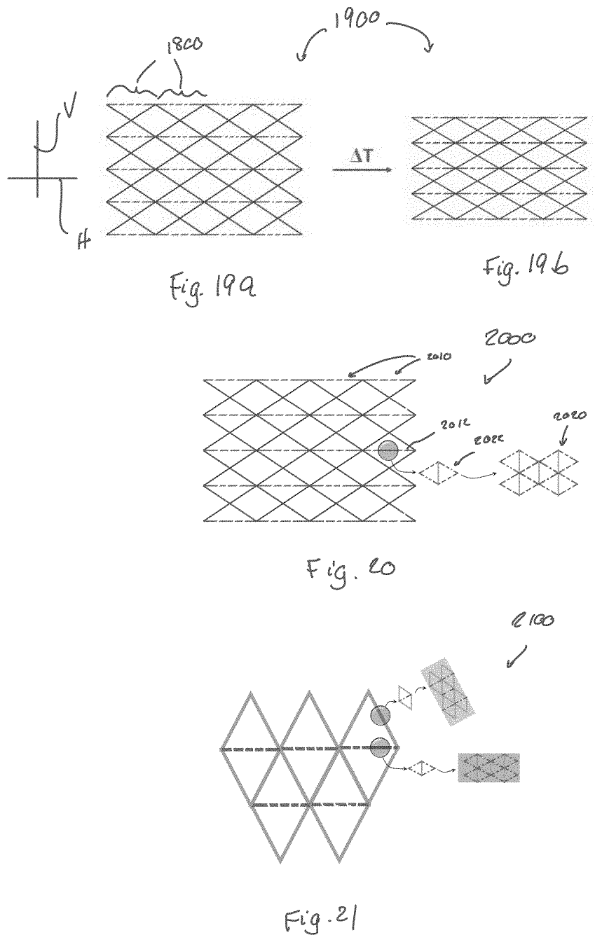

FIGS. 22a to 25b illustrate a plurality of different metaensembles 2200, 2300, 2400, 2500, 2600 each shown in undeformed (FIGS. 22a, 23a, 24a, 25a, 26a) and deformed configurations (FIGS. 22b, 23b, 24b, 25b, 26b). Each of those metaensembles 2200, 2300, 2400, 2500 exhibits a shrinkage upon a temperature increase and may be made by assembling a plurality of the metaunits 1800 described above with reference to FIG. 18.

The metaunit 2200 of FIG. 22a includes groups a metaunits 1800 disposed in parallel about horizontal axes H; the groups circumferentially distributed about a central axis R normal to the vertical axes H. The metaensemble 2200 may exhibit shrinkage in a direction parallel to the central axis R.

The metaunit 2300 of FIG. 23a includes groups of metaunits 1800 disposed in series about vertical axes V; the groups circumferentially distributed about a central axis R normal to the vertical axes V. The metaensemble 2300 may exhibit shrinkage in a radial direction parallel relative to the central axis R. This may be referred to as circumferential shrinkage.

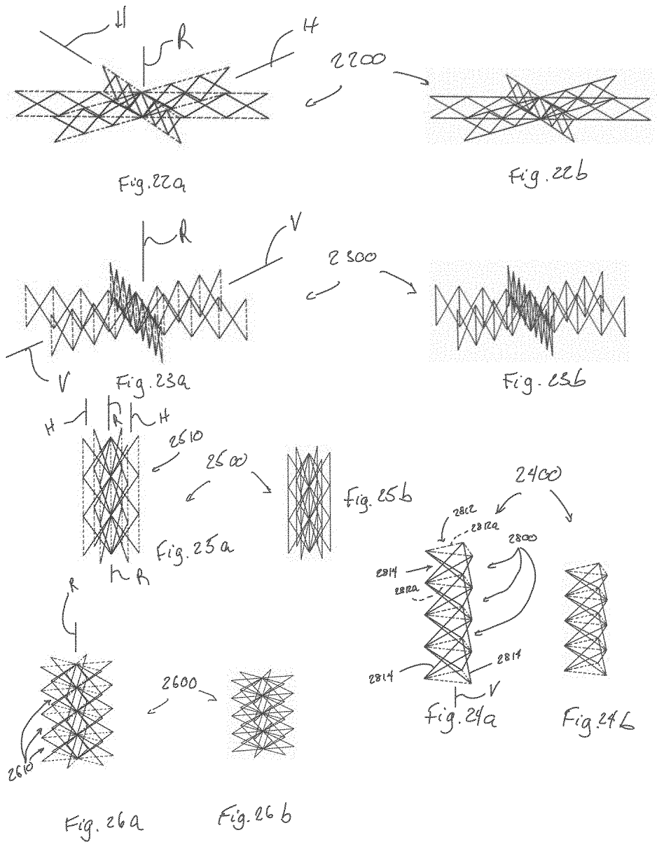

The metaunit 2400 of FIG. 24a includes groups of three-dimensional metaunits 2800. The metaensemble 2400 includes a plurality of the metaunits 2800 disposed in series about a vertical axis V. Each metaunits 2800 may include a frame 2812 having upper and lower sections 2812a of a triangular shape. The frame sections 2812 are shown in dashed lines in FIGS. 24a, 24b. Cores 2814 may include each six members 2814a. Each corners of the upper frame sections 2812a may be connected to two opposite corners of the lower frame sections 2812a via two of the six frame members 2814a. It is understood that other shapes are contemplated, such as square, circle, and so on. The disclosed metaunit 2400 may exhibit a shrinkage along the vertical axis V upon a temperature increase.

Referring now to FIGS. 25a and 25b, another embodiment of a metaensemble is shown at 2500. The metaensemble 2500 includes plurality of three-dimensional metaunits 2510. The metaunits 2510 includes frames shown in dashed line and cores shown in solid lines. The metaunits 2510 may be distributed circumferentially about a central axis R. The metaensemble may exhibit a circumferential shrinkage upon a temperature increase.

Referring now to FIGS. 26a and 26b, another embodiment of a metaensemble is shown at 2600. The metaensemble 2600 includes plurality of three-dimensional metaunits 2610. The metaunits 2610 includes frames shown in dashed line and cores shown in solid lines. The metaunits 2610 may be distributed circumferentially about a central axis R. The metaensemble may exhibit a vertical shrinkage in a direction parallel to the central axis R upon a temperature increase.

It is understood that each configurations depicted above with reference to FIGS. 18a to 26a may use any of the metaunits disclosed herein above that may exhibit an increase in a control dimension (e.g., height) upon a temperature increase.

In one embodiment, the cells, or portions thereof, as disclosed in international patent application publication no. WO2018/227302, the entire content of which is incorporated herein by reference, may be incorporated in whole or in part with the metamaterials as described herein.

For producing a metaensemble including a plurality of metaunits and defining a sequence code, one or more geometric characteristics of the target shape are determined; the determined geometric characteristics of the target shape are translated into geometric characteristics of each of the plurality of metaunits forming the metaensemble; a change of shape of the metaensemble is determined so that the metaensemble morphs to the target shape upon exposure to the temperature condition; material and complementary geometric properties of each of the metaunits are determined based on the determined change of shape of the metaensemble; and the metaensemble is manufactured based on the determined sequence code.

In the embodiment shown, determining the geometric shape includes modeling the target shape as a target domain with a central axis with upper and lower boundaries. As shown, translating the determined characteristics includes determining lengths of each of the metaunits based on distances between the upper and lower boundaries. In a particular embodiment, determining the change of shape of the metaensemble includes determining distances between the central axis of the target domain and a central axis of the metaensemble being undeformed. In a particular embodiment, determining the material and the complementary geometric properties includes determining a change of shape each of the metaunits must present for the metaensemble to morph to the target shape and translating the determined change of shape in the material and complementary geometric properties. In the embodiment shown, each of the metaunits has a frame and a deformable member, the deformable member having a coefficient of thermal expansion (CTE) greater than that of the frame, the frame having a Young's modulus greater than that of the deformable member, determining the material characteristics includes determining the CTE and the Young's modulus of each of the deformable member and the frame of each of the metaunits.

Underpinned by three distinctive notions (FIG. 1), the present framework may deterministically predict and precisely impart morphing into a single-piece metamaterial made of passive solids upon a change in temperature. The shape matching of the phenotype to a target domain may be accurately controlled in space through a decoded BB sequence. The constitutive solids may be passive, yet their topological arrangement into our metaunit may form aperiodic aggregates that may yield reconfigurations of broad geometric diversity.

The kirigami concepts here disclosed may not require chemical strategies but rather use geometric strategies applicable to several pairs of off-the-shelf solids including metals. If needed, the selection of the base materials can address the requirement of robustness to fluctuating thermal stress. In addition, the rational manipulation of their geometry, such as the size of the BB groove and the offset of the flexural hinge, may allow to calibrate both the rate of deformation and the temperature range within which the response occurs. This geometric tuning may offer significant freedom to generate desired types of response, including both sudden and smooth deformation, which could be gradually dispensed even over a large temperature span.

There are a number of potential applications for shape-matching materials across multiple sectors, especially where folding, packaging, and conformational changes are important requirements to meet, such as self-reconfigurable medical devices, drug delivery systems, autonomous soft robotics, and conformable stretchable electronics. The advantages of the concepts here introduced may be capitalized in two primary applications. The first may target repeated and reversible reconfigurability in extreme climates on Earth and in space. Here the transportation of components is typically required in a flat configuration, the deployment is to occur in-situ, such as unfolding shelters in unsafe settings or reconfigurable antennas in space, and reconfigurability may entail multiple loops of closure and opening, each controlled by temperature cycles. In these conditions, shape memory polymers and other active materials may not be the best fit, not only because their response is typically irreversible, but also because thermomechanical cycles may steadily decrease their performance. The second application may be thermal management. Besides shape morphing, the disclosed concepts may be programmed to feature adaptive change in their out-of-plane porosity in response to temperature change. The transformation from a fully solid to a fully porous state through temperature change may bring about a large area of voids for heat exchange, conditions that can become an asset for cooling and thermal regulation.

Overall, the disclosed framework may engage a fine interplay between geometry and mechanics of metaunits to enact morphing in response to temperature. It may require neither manipulation of constituent compositions nor chemical processes. It may predict local and global morphing, as well as reconfigure the morphology of aperiodic architectures into predefined targets. Reversibility through temperature may be one of its assets, along with the passive nature of the constituents, and the elimination of external power and control. A large design freedom to tune the thermal response (type, magnitude and rate of deformation) may be at hand through manipulation of the internal architecture. Other pairs of passive solids including metals may be used, as long as they offer a suitable distinction in CTE. Purposely implemented with simple yet efficient means of fabrication, the disclosed platform may be well-suited to other technologies, e.g. multi-material 3D printing, may offer routes for upscaling and downscaling, and may be also extended to active materials and other stimuli.

More detail may be found in publication: Liu, L., Qiao, C., An, H. et al. Encoding kirigami bi-materials to morph on target in response to temperature. Sci Rep 9, 19499 (2019), https://doi.org/10.1038/s41598-019-56118-2, the entire content of which is incorporated herein by reference.

Embodiments disclosed herein include:

A. A metamaterial configured to reversibly deform when exposed to a temperature condition, comprising a plurality of metaunits interconnected with one another to form a metaensemble, each of the metaunits having a frame and a core attached to the frame, a portion of the core free of connection with the frame to allow relative movement therebetween, one of the frame and the core having a Young's modulus greater than that of the other and having a coefficient of thermal expansion less than that of the other of the frame and the core, the metaensemble having a sequence code defining a target shape of the metaensemble, the sequence code including at least one geometric characteristic and at least one material characteristic of each of the frame and the core, the metamaterial with the sequence code being reversibly deformable from an initial shape to the target shape upon being exposed to the temperature condition and back from the target shape to the initial shape upon withdrawal of the temperature condition.

B. A metaunit for forming a metamaterial, comprising a frame and a core secured to the frame, a portion of the core free of connection with the frame to allow relative movement therebetween, one of the frame and the core having a Young's modulus greater than that of the other and having a coefficient of thermal expansion (CTE) less than that of the other of the frame and the core, the metaunit reversibly deformable from a first position to a second position upon application of a temperature condition and from the second position to the first position upon withdrawal of the temperature condition, a deformation of the metaunit upon application of the temperature condition different than that of both the frame and the core being separated from one another.

Embodiments A and B may include any of the following elements, in any combinations:

Element 1: the cores are secured to the frames solely at extremities of the cores. Element 2: the frames at least partially enclose the core. Element 3: the cores at least partially enclose the frames. Element 4: the geometric properties contained within the sequence code includes dimensions of the frame and dimensions of the core. Element 5: the material properties contained within the sequence code includes the Young's modulus and the CTEs of the frames and the cores. Element 6: a ratio of a CTE of the core over the CTE of the frame is at least 10. Element 7: a ratio of the Young's modulus of the frame over the Young's modulus of the core is at least 10. Element 8: at least one of the metaunits is asymmetrically deformable upon exposure to the temperature condition. Element 9: at least one of the metaunits is symmetrically deformable upon exposure to the temperature condition. Element 10: the temperature condition is an increase in an ambient temperature. Element 11: the frame has a greater Young's modulus than that of the core and a CTE less than that of the core. Element 12: the frame includes upper and lower frame members connected to one another by the core. Element 13: the frame has a higher CTE than that of the core, a control dimension of the metaunit decreasing upon an increase in temperature. Element 14: the frame has a lower CTE than that of the core, a control dimension of the metaunit increasing upon an increase in temperature.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure, and such modifications are intended to fall within the appended claims.

1. A metamaterial configured to reversibly deform when exposed to a temperature condition, comprising a plurality of metaunits interconnected with one another to form a metaensemble, each of the metaunits having a frame and a core attached to the frame, a portion of the core being free of connection with the frame to allow relative movement therebetween, one of the frame and the core having a Young's modulus greater than that of the other and having a coefficient of thermal expansion less than that of the other of the frame and the core, the metaensemble having a sequence code defining a target shape of the metaensemble, the sequence code including at least one geometric characteristic and at least one material characteristic of each of the frame and the core, the metamaterial with the sequence code being reversibly deformable from an initial shape to the target shape upon being exposed to the temperature condition and back from the target shape to the initial shape upon withdrawal of the temperature condition.

2. The metamaterial of claim 1, wherein the cores are secured to the frames solely at extremities of the cores.

3. The metamaterial of claim 1, wherein the frames at least partially enclose the core.

4. The metamaterial of claim 1, wherein the cores at least partially enclose the frames.

5. The metamaterial of claim 1, wherein the geometric properties contained within the sequence code includes dimensions of the frame and dimensions of the core.

6. The metamaterial of claim 1, wherein the material properties contained within the sequence code includes the Young's modulus and the CTEs of the frames and the cores.

7. The metamaterial of claim 1, wherein a ratio of a CTE of the core over the CTE of the frame is at least 10.

8. The metamaterial of claim 1, wherein a ratio of the Young's modulus of the frame over the Young's modulus of the core is at least 10.

9. The metamaterial of claim 1, wherein at least one of the metaunits is asymmetrically deformable upon exposure to the temperature condition.

10. The metamaterial of claim 1, wherein at least one of the metaunits is symmetrically deformable upon exposure to the temperature condition.

11. The metamaterial of claim 1, wherein the temperature condition is an increase in an ambient temperature.

12. The metamaterial of claim 1, wherein the frame has a greater Young's modulus than that of the core and a CTE less than that of the core.

13. A method of producing a metamaterial configured to reversibly deform from an initial shape to a target shape upon exposure to a temperature condition, the metamaterial including a metaensemble formed of a plurality of metaunits each having a frame and a core attached to the frame, the method comprising:
obtaining one or more geometric characteristics of the target shape;
determining a sequence code of the metaensemble such that the metamaterial deforms to the target shape upon application of the temperature condition, the sequence code including at least one geometric characteristic and at least one material characteristic of each of the metaunits of the metaensemble, wherein a portion of the core of the metaunits being free of connection with the frame to allow relative movement therebetween, one of the frame and the core having a Young's modulus greater than that of the other and having a coefficient of thermal expansion less than that of the other of the frame and the core; and
manufacturing the metamaterial based on the determined sequence code.

14. The method of claim 13, wherein determining the sequence code includes:
a) selecting first values of the sequence code;
b) obtaining a model of the metamaterial based on the first values of the sequence code;
c) simulating a deformation of the model of the metamaterial upon exposure to the temperature condition;
d) determining second values of the sequence code in function of a difference between the simulated deformation of the model of the metamaterial and the target shape; and
e) repeating steps b) to d) until the simulated deformation of the model matches the target shape.

15. The method of claim 13, wherein determining the sequence code includes determining Young's moduli, CTEs, and dimensions of each of the frames and the cores of each of the metaunits.

16. The method of claim 13, wherein obtaining one or more geometric characteristics of the target shape includes modeling the target shape as a target domain with a central axis with upper and lower boundaries.

17. A metaunit for forming a metamaterial, comprising a frame and a core secured to the frame, a portion of the core free of connection with the frame to allow relative movement therebetween, one of the frame and the core having a Young's modulus greater than that of the other and having a coefficient of thermal expansion (CTE) less than that of the other of the frame and the core, the metaunit reversibly deformable from a first position to a second position upon application of a temperature condition and from the second position to the first position upon withdrawal of the temperature condition, a deformation of the metaunit upon application of the temperature condition different than that of both the frame and the core being separated from one another.

18. The metaunit of claim 17, wherein the frame includes upper and lower frame members connected to one another by the core.

19. The metaunit of claim 18, wherein the frame has a higher CTE than that of the core, a control dimension of the metaunit decreasing upon an increase in temperature.

20. The metaunit of claim 18, wherein the frame has a lower CTE than that of the core, a control dimension of the metaunit increasing upon an increase in temperature.
